(19) United States
(12) Patent Application Publication
(10) Pub. No.: US 2002/0155422 A1
(43) Pub. Date: Oct. 24, 2002

(54) Methods for analyzing dynamic changes in cellular informatics and uses therefor

(76) Inventors: Donald E. Ingber, Boston, MA (US); Sul Huang, Boston, MA (US)

Correspondence Address: TESTA, HURWITZ & THIBEAULT, LLP, HIGH STREET TOWER, 125 HIGH STREET, BOSTON, MA, 02110, US

(21) Appl. No.: 09/985,963

(22) Filed: Oct. 19, 2001

Related U.S. Application Data
(60) Provisional application No. 60/242,009, filed on Oct. 20, 2000.

(51) Int. Cl. G06F 17/60; C12Q 1/00; C12Q 1/68; G06F 19/00; G01N 33/48; G01N 33/50

(52) U.S. Cl. 435/4; 435/6; 702/20; 705/3

(57) ABSTRACT

Methods are provided for analyzing dynamic changes in cellular processes and for representing cellular processes as dynamic signatures or phase portraits. Methods of the invention are useful for comparing cellular processes and providing diagnostic and prognostic information. Methods of the invention are also useful for identifying important molecular components of cellular processes, for identifying targets for drug development, and in assays for identifying drug candidates and evaluating drug effectiveness.

### RELATED APPLICATIONS

This application claims priority to, and the benefit of U.S. Ser. No. 60/242,009 filed Oct. 20, 2000, the disclosure of which is incorporated by reference herein in it entirety.

### GOVERNMENT SUPPORT

Work described herein was supported, in part, by NIH Grant CA58833. The U.S. Government has certain rights in the invention.

### FIELD OF THE INVENTION

The invention relates generally to methods for identifying and analyzing time-dependent patterns of genome-wide cell activities, to methods for producing dynamic signatures and phase portraits to represent genome-wide patterns of gene or protein activity, and to methods that rely on use of dynamic signatures and phase portraits to identify mechanistically relevant molecules that contribute to changes in cell behavior state. In particular, the invention is related to disease diagnostic and prognostic methodologies and to drug target identification and drug screening assays based on methods for analyzing and representing time-dependent changes in cell-wide activity.

### BACKGROUND OF THE INVENTION

The recent development of massively-parallel methods for analyzing patterns of gene activity in a cell or tissue has opened up new avenues for studying cellular behavior that may be critical for drug discovery and the understanding of disease. However, conventional studies of gene expression assume that there is a simple relationship between the genome and a disease or a drug response. A typical analysis relies on generic pattern-recognition methods to 1) compare gene expression in different cell states or tissues and identify differentially expressed genes, or 2) cluster genes or gene expression profiles (patient samples) that show similar expression characteristics to identify the characteristic modes in temporal profiles or to define distinct pathological conditions. While such an analysis may be useful for some diagnostic purposes, it is based solely on a generic statistical analysis. Such methods of analysis fail to take into account specific features inherent to the complexity of information processing by living cells, and thus fail to identify functional or causal relationships between genes involved in a biological process of interest.

Similarly, conventional approaches to predict cell behavior typically are based on identification of “molecular markers” that statistically correlate with a particular cellular outcome. However, such approaches do not reveal the complex molecular mechanisms that cause cells to switch between distinct behavioral states and hence, determine cellular fate. Therefore, such approaches can provide only a crude prediction of cellular outcome, and fail to provide the sophisticated information that would be useful to predict cellular fate at an early stage during the transition between different cellular behavior states, such as during the response to a drug, drug candidate, or toxin, or during the switch between health and disease.

There is therefore a need in the art for materials and methods for collecting and processing large amounts of cellular information in order to identify and exploit complex molecular interactions involved in important cellular processes that involve transitions between distinct cellular behavioral states.

### SUMMARY OF THE INVENTION

The invention provides methods and materials for identifying and representing dynamic patterns of molecular change that are characteristic of specific cellular processes. According to the invention, cell activity profiles (e.g. profiles of gene expression or protein activation) are analyzed as a function of time in order to identify patterns that reflect important functional and mechanistic relationships between genes and/or gene products. Methods of the invention involve analyzing a large number of cellular characteristics and providing functionally relevant information relating to a cellular process of interest. For example, methods of the invention are useful to identify one or more genes that may cause a switch to a disease-promoting cell state but that are not expressed in the final diseased state. Such genes would not be detected using conventional static profile comparisons.

Methods of the invention are based on an analysis of temporal changes in patterns of cell activities measured during cellular processes after a distinct stimulus. According to the invention, characteristic pattern changes result from the existence of underlying molecular wiring networks within the cell. However, analysis methods of the invention do not require that the wiring network be known or understood. Indeed, the invention does not require that specific functions be pre-assigned to individual genes or proteins in a cell, nor that the specific architecture of the underlying network of molecular interactions be inferred from the dynamics of cell-wide activity profiles. Rather, the present invention exploits the observation that a cell's underlying regulatory network is reflected in the dynamic properties of cell-wide molecular activities during a cellular process. According to the invention, a time-dependent analysis of cell activities provides useful insight into the functional properties and mechanistic relationships within the underlying regulatory network, even though the identity of the individual components may not be known.

The invention provides methods for representing dynamic changes in a number of cellular activities such as gene or protein expression. According to the invention, a complex set of molecular changes associated with a cellular process is represented as a dynamic signature that is characteristic of the process. A typical dynamic signature is based on time-dependent molecular changes that are associated with a transition between distinct, stable cellular behavioral states. In a preferred embodiment of the invention, a chosen cellular transition process has a unique dynamic signature. A preferred dynamic signature is a representation (e.g. a mathematical, an electronic, a data set, or a graphic representation) of time-dependent changes in multiple, mechanistically-linked variables (molecular activities) that mediate a cellular transition process, rather than single molecular markers or artificially clustered groups of markers. In a particularly preferred embodiment of the invention, a dynamic signature is expressed as a phase portrait, providing a graphical representation of cellular activity changes that are characteristic of a given cellular process or event.

According to preferred embodiments of the invention, useful cellular information includes genome-wide changes in gene expression, changes in protein expression and/or protein activity. However, other indicators of cellular activity can also be assayed and used to generate a dynamic signature for a particular cellular process. Useful indicators of cellular activity include cellular molecules or molecular components of cellular activity including the levels or identity or modifications of nucleotides (including DNA, and RNA such as tRNA, rRNA, mRNA), peptides, carbohydrates, lipids, metabolic intermediates, and intra or extra cellular salts and other solutes.

Preferred cellular processes are transition processes with a defined start-point, such as a cellular response to a drug, toxin, pathogen, or other external stimulus. Particularly preferred cellular events also have a defined end-point, such as a cellular transition from one stable cell behavioral state to another stable cell state. Preferred cellular transitions include transitions from a healthy state to a diseased state, from a diseased state to a healthy state, from an undifferentiated state to a differentiated state, from a differentiated state to an undifferentiated state, from one differentiated state to another differentiated state, and among growth, differentiated, apoptotic, motile, contractile, quiescent and senescent states. According to the invention, cellular processes can be measured in vivo or in vitro, including in a cell culture, a tissue culture, a tissue, an organ, or an organism.

Distinguishing meaningful information from the volumes of data that can be generated with genome-wide gene and protein profiling techniques is an important aspect of the invention. Analysis methods of the invention are based on “cellular informatics”—how living cells actually process information. The invention provides technology that circumvents current limitations and leads directly to the identification of genes and proteins that are mechanistically relevant to a given cellular process, and hence, prime targets for therapeutic intervention in the context of a disease. This technology links novel cell system-based modes of data acquisition to proprietary software tools, and represents a generic approach that can be applied to any disease process or drug screening program that involves changes in cell regulation.

According to the invention, cells have built-in information processing rules that are based on internal wiring of molecular signaling pathways within complex interdependent networks. The existence of these networks imposes particular dynamic constraints on gene and protein signaling activities. The present invention makes use of the growing understanding of the mathematics of dynamic networks and of the biology of cell regulation to extract knowledge about how cells respond to regulatory signals, pathological influences, and pharmacological perturbations.

Accordingly, in one aspect, the invention provides an algorithmic approach to identify the precise temporal series of gene and protein switches that drive changes in cell and tissue function, much like decoding the time-dependent sequence of numbers that opens a combination lock. Thus, all possible patterns of cell activity (e.g. all patterns of gene or protein activity) can be represented on a topological landscape of temporal cell-state space, and rather than just referring to a few points on the landscape, the invention uncovers entire pathways in the landscape. Using this technology, dynamic signatures are identified within genome-wide gene and protein activity profiles that are prognostic for cell switching between different behavioral states, such as the transition between different stem cell lineages, from growth to apoptosis, or between malignancy and the normal state.

Dynamic signatures of the invention can be applied to predict cellular fate, and as the dimensionality of the information used for the prediction increases from one to many variables, the predictive power of the information increases. Thus, according to the invention, as additional genes or proteins activities are included in the analysis, the time-dependent patterns of cellular activity are refined, and the predictive power of these patterns is increased.

In addition, predictive dynamic signatures of the invention can be further processed iteratively, mathematically, or electronically (for example on a computer system) to identify specific genes and molecules that contribute most significantly to a physiological or pathological response that is being studied. In a preferred embodiment, a representative or reference dynamic signature or phase portrait is identified based on a complete data set of cellular activity measured over time. One or more dynamic signatures or phase portraits are also generated from subsets of the data (e.g. using between 50% and 100% and preferably about 60% or 80% of the data set). The representations generated based on the data subsets are compared to the reference representation. If they are similar, the subset contains most or all of the important molecular components of the cellular process being analyzed. This process can be repeated iteratively until a smaller set of data is identified that is responsible for the dynamic signature or phase portrait of the cellular process. This smaller set represents the molecular components that are important for the cellular process. In one embodiment, one or several individual molecules (e.g. genes) are identified. Some of these are mechanistically important for the cellular process in that they are causative, others are tightly associated with the process, but not causative. Such individual molecules or subsets of molecules are useful as drug targets for drug development programs. Alternatively, these smaller subsets can be used in subsequent analyses to generate dynamic signatures or phase portraits that are useful to evaluate or monitor cellular processes for different applications described herein (such as drug screening assays). According to the invention, subsets can be chosen randomly or based on dimensionality reduction using for example a clustering method or a principal component analysis. According to one iterative method of the invention, one or more subsets of previously chosen or identified subsets are further analyzed to further narrow the number of data points required to generate a representative dynamic signature or phase portrait.

Accordingly, methods and compositions of the invention are useful for disease diagnosis and prognosis (e.g. for predicting disease progression), for automated identification of drug targets for multi-gene diseases (e.g., heart disease, hypertension, stroke, cancer, arthritis, and other multi-gene diseases), for the prediction of drug and toxin effects, for the prediction of clinical response to therapy, for the identification and control of differentiation paths for stem cell-based therapies, for the development of therapies that involve the switching of cell states, such as switching growing cancer cells to quiescent, differentiated, or apototic cells for developing cell-based disease model systems (e.g., atherosclerosis, angiogenesis, stem cell biology, osteoporesis etc.), and for in silico replacement of animal testing and existing cumbersome methods used for lead target validation in drug development.

An important aspect of the invention is the use of dynamic signatures to identify drugs that target multiple molecules. According to the invention, a dynamic signature can represent multiple molecular changes in a cell and can be used to screen candidate drugs to identify those that affect multiple molecular targets. This aspect of the invention is particularly useful, because many diseases involve multiple molecular changes.

Methods and materials of the invention also extend to computer databases and software programs for generating, storing, retrieving, accessing, and analyzing information of the invention related to cellular activity. Data relating to cellular activity profiles, dynamic signatures, phase portraits, and other forms of representation can be electronically stored and analyzed. Accordingly, analysis methods of the invention can be stored electronically and implemented in a computer system.

In another aspect, the invention provides drugs that are identified according to the methods of the invention. In one embodiment, a drug is selected from a series of candidate drugs using screening assays of the invention. In an alternative embodiment, a drug is designed based on the identity of one or more drug targets that were identified according to methods of the invention.

### BRIEF DESCRIPTION OF THE DRAWINGS

FIG. 1a shows an embodiment of a 4-gene network.

FIG. 1b shows cell state transitions associated with the gene network of FIG. 1a.

FIG. 1c shows basins of attraction associated with the gene network of FIG. 1a.

FIG. 2 shows the gene activity values for each gene over time for a cellular process involving the 4-gene network embodiment of FIG. 1.

FIG. 3 shows an embodiment of a distance matrix of inter-pattern distances for the 4-gene network of FIG. 1.

FIG. 4a shows a state space with trajectories (S1, S2, S3) for cell state transition processes between the cell states A, B, C and the various distance measures D(t)

FIG. 4b shows a theoretical phase portrait for a single cellular process for a transition between two attractors (A to B) after a stimulus.

FIG. 4c shows a theoretical graph for the temporal behavior of the inter-trajectory distance DT(t) exhibiting convergence (A to B) or divergence (A to C versus A to B).

FIG. 5 shows an embodiment of phase portraits involving the 4-gene network of FIG. 1. The left panel represents a single cellular process, the right panel represents two different cellular processes.

FIG. 6 shows a phosphoimager scan of a gene filter.

FIG. 7 shows the temporal behavior of an intratrajectory distance for hematopoietic differentiation induced by DMSO and hematopoietic differentiation induced by retinoic acid.

FIG. 8 shows overlaid phase portraits generated using subsets of gene expression data representing a switch from a proliferation state to a differentiation state induced by retinoic acid.

FIG. 9 shows phase portraits for gene expression level changes for known cellular processes, the left panel shows induction of growth in quiescent fibroblasts, the right panel shows induction of hematopoietic differentiation in proliferating HL-60 cells.

FIG. 10 shows the robustness of phase portraits using gene expression level changes for induction of growth in fibroblasts, panel A shows a phase portrait based on approximately 6000 genes, panel B shows a series of phase portraits based on random selections of 80% of the approximately 6000 genes, panel C shows a series of phase portraits based on random selections of 60% of the approximately 6000 genes.

FIG. 11 shows an embodiment of a flow diagram of methods for generating a phase portrait of a cellular process according to the invention.

FIG. 12 shows an embodiment of methods of the invention for analyzing complex cellular information and generating databases containing representations of complex cellular processes.

### DETAILED DESCRIPTION OF THE INVENTION

The invention provides methods and materials related to the detection, identification, and understanding of molecular mechanisms underlying cellular processes, such as a transition from one cell behavioral state to another, or a cellular response to a drug or toxin, or processes involving the concerted action of multiple cells, such as inflammation, immune response, angiogenesis, malignant transformation, tumor progression or tissue regeneration. Specifically, the invention provides methods and materials for performing genome-wide or cell-wide studies of molecular activity during a cellular process whether within a single type of cell or a complex tissue. According to the invention, time-dependent patterns of cellular activity are detected by observing and analyzing the activity of a plurality of cellular markers, preferably genes or proteins, throughout the duration of a cellular process that mediates a transition from one cellular state to another. According to the invention, subsets of functionally relevant cellular markers are identified by analyzing the patterns of time-dependent change in genome-wide cellular activity. Cellular activity can be measured using any one of a number of different types of cellular markers, including DNA modification, mRNA expression, protein expression, protein activation, post-translational modification, subcellular localization, lipid metabolism, carbohydrate chemistry, and other molecular markers. Accordingly, an analysis of genome-wide or cell-wide activity includes numerous markers of one or more different types.

An important feature of the invention is the analysis of dynamic patterns of cellular activity. By focusing on patterns of activity, the invention provides methods for rapidly reducing the complexity of cellular information into a form that is useful for identifying important genes, screening drug candidates, evaluating the effectiveness and toxicity of lead drug candidates, and other applications involving an analysis of complex cellular processes.

Methods of the invention provide useful information from a global pattern of change in cellular activity without focusing on each component of the cellular activity (e.g. each gene or protein). The invention does not require the tedious elucidation of the entire or partial wiring diagram of a cell regulatory or signaling pathway. Instead, functional cellular data is directly linked to a biological process by analyzing the patterns of cellular activity that reflect the existence of an underlying dynamic network governing the manner in which a cell processes information. In a preferred embodiment of the invention, the activity of cellular markers is analyzed using a mathematical algorithm to identify a pattern of changes in molecular activities that is characteristic of the cellular process. Such a pattern is referred to as a dynamic signature for the transition between different cellular behavioral states. In a preferred embodiment of the invention, a dynamic signature is represented graphically as a phase portrait characteristic of the molecular changes that occur as a cellular event unfolds. Dynamic signatures or phase portraits are useful to predict cell fate, for disease diagnosis and prognosis, and as indicators of different cellular or tissue processes such as toxicity or drug action. In other embodiments of the invention, a dynamic signature or phase portrait is used as a base from which to identify particular molecules and hence, potential drug targets or diagnostic markers, that are mechanistically involved in the transition between different cellular states.

According to the invention, the range of molecular events that are possible for a given cell is constrained by the structural, functional and regulatory interactions between different genes, gene products, proteins and other molecular and chemical components of the cell. In particular, the nature of the genes expressed by a cell, the functional properties of the proteins present in a cell, and the network of regulatory interactions between all of these molecular components determine the manner in which the cell responds to an external stimulus or transitions from one cell state to another cell state. However, despite these constraints on cellular activity, the complexity of the interactions between the many different molecules present inside a cell is poorly understood. Typical studies of a cellular process focus on a specific molecule or a specific subset of molecules whose expression or activity is highly correlated with a specific outcome of the cellular process, such as the transition from a normal to a malignant behavioral state or the behavioral response of a cell to a drug. These studies ignore the larger cellular context of these molecules and their structural, functional, and regulatory interactions with other cellular components during the cellular process being analyzed, and thus, they fail to address the transition between cell states as an integrated whole. Accordingly, these studies provide only a crude picture of molecular activities that are associated with a particular biological process, and are poorly predictive of a cellular outcome or of a cellular response to an external stimulus such as a drug treatment. By ignoring the complex networks of molecular interactions inside a cell, and by focusing on a qualitative and static analysis of only one or a subset of molecules, typical studies fail to explore and exploit the wealth of information that is available in a cellular system.

The invention provides methods and materials for analyzing the genome-wide information that can be assayed during a cellular process that involves a transition between distinct cell behavioral states. Although the available information is complex, methods of the invention take advantage of the fact that cellular systems are constrained by structural, functional, and regulatory interactions between the cell's molecular components that effectively form a basic wiring diagram determining the actual range and patterns of cellular activities that are possible.

An example of the nature of these constraints and how-they arise is provided in a simplified example of a “model” cell containing only 4 interacting binary molecules (“genes”) using a boolean network formalism. However, the same type of dynamics can arise in continuous value networks, in which molecular interactions, such as gene-gene interactions or protein-protein interactions, are described by sigmoidal kinetics as typically observed in biological systems. Although the 4 gene example shows how the cell's internal wiring diagram constrains its possible behavioral responses, an important feature of the present invention is that it does not require the tedious elucidation of the entire or partial wiring diagram of the cell in order to predict these responses or identify molecules that contribute to the response. Instead, functional cellular data is directly linked to a biological process by analyzing dynamic changes in the pattern of genome-wide cellular activities during the transition between different cellular behavioral states. The typical form of the changes reflects the existence of the underlying wiring diagram.

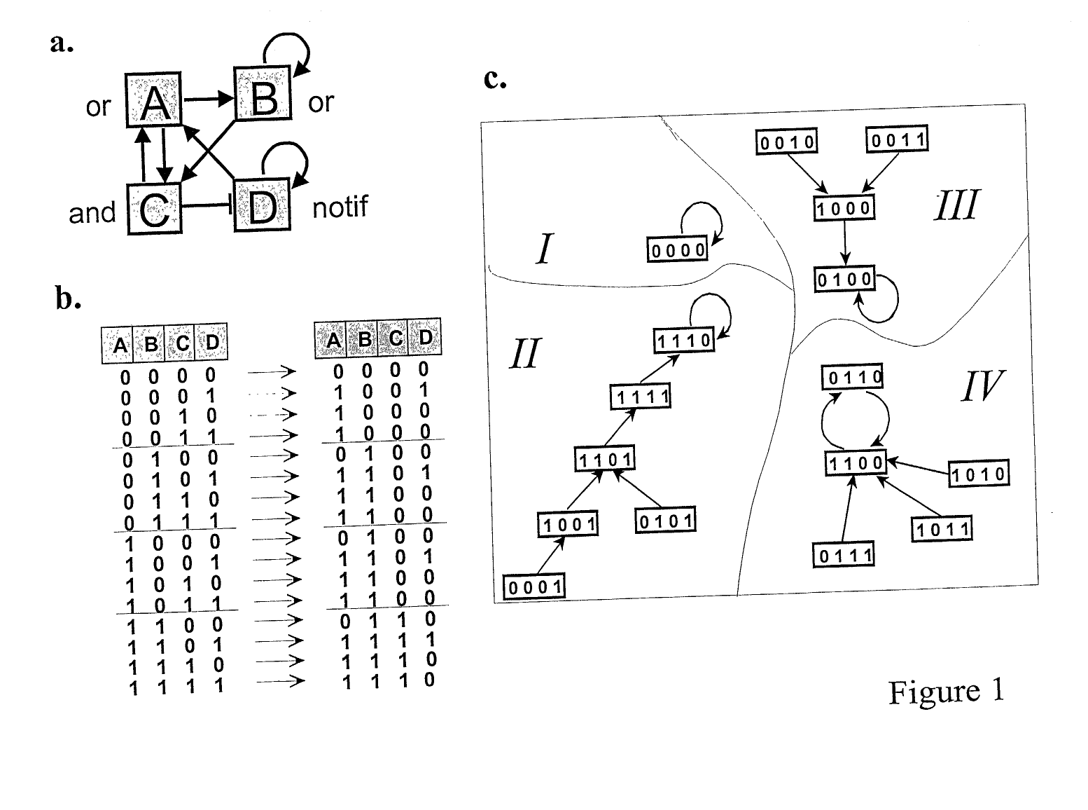

In the four molecule network shown in FIG. 1, the cell state space is 4 dimensional, with the activity of each of gene A-D being a component of the vector. The table shown in FIG. 3 shows the vector components as a function of time. At each time point, the cell state vector is defined collectively by the activity level of each of genes A-D. This example illustrates the basic idea of a boolean network of interacting genes or proteins. This example illustrates a type of wiring network that represents the basic structural and functional properties of a cell and determines the possible patterns of gene expression and molecular activities. However, according to methods of the invention, this wiring network does not need to be known. Indeed, methods of the invention are based on an analysis of temporal changes in the pattern of genome-wide cell activities (e.g. patterns of gene or protein activity) that result from the existence of an underlying cellular wiring network, but the analysis methods of the invention do not require that the wiring network be known or understood. The analysis method of the invention is based on novel scientific insights into the generic structural and dynamic properties of cellular regulatory networks: 1) The cellular information processing network (molecular interaction wiring diagram) is sparsely connected. 2) The global dynamic that ensues is that the state vector moves relatively “smoothly”, thus allowing reduction of dimensionality of its trajectory. 3) Due to the constraints inherent in the cellular network of regulatory interactions, the number of possible trajectories is relatively small compared to the vast number of interacting elements (genes, proteins, other molecules and chemicals) and possible state vectors.

In the highly simplified model example shown in FIG. 1A, the network consists of the N=4 binary molecules (genes, proteins, etc.) A, B, C and D in which every molecule can take the values 1(=ON) or 0(=OFF). In this network every molecule has two inputs. A gene can receive input from itself (as is the case for genes B and D). The network wiring diagram consists of the topology (which defines which gene is connected to which other gene) and the boolean functions assigned to each individual molecule (which defines how the gene responds to its two inputs). Boolean functions are standard functions that determine how the value (0,1) of the inputs collectively determine the value of the output. That is, the function determines the activity state of the molecule to which the boolean function is assigned. For the two input situation, boolean functions have specific names such as ‘and’, ‘or’, ‘notif’ and others. For instance, gene A gets inputs from C and D (FIG. 1A) and has the boolean function ‘or’, implying that the output to genes C and B (the activity of A) will be 1 (ON) if either one C or D is ON. Gene D also gets inputs from C and D (FIG. 1A) but has the boolean function ‘notif’, implying in this example that the output to genes A and D (the activity of D) will be 1 (ON) if D is ON, unless C is ON. The ON(1) and OFF(0) status of each gene collectively define a gene activity profile which changes upon updating in discrete time. The state transition table in FIG. 1B shows the 16 initial states and the transitions from state to state as constrained by the network wiring diagram in FIG. 1A. For example, if a cell is in the state 0001, meaning that only molecule D is ON, the cell must transition to state 1001 (both molecules A and D are ON) due to the action of gene D on gene A, according to the wiring diagram of FIG. 1A. This transition is shown in the second line of the table in FIG. 1B. Furthermore, a cell in state 1001 transitions to state 1101 (A, B and D are ON) due to the action of A on B, according to the wiring diagram of FIG. 1A. This transition is shown in line 10 of the table in FIG. 1B. A cell in state 1101 transitions to state 1111, (A, B, C and D are all ON), the change being due to the action of A and B on C, according to the wiring diagram of FIG. 1A. This transition is shown on line 14 of the table in FIG. 1B. Finally, a cell in state 1111 transitions to state 1110 (A, B and C are ON, and D is now OFF) due to the action of C on D, according to the wiring diagram of FIG. 1A. This transition is shown on line 16 of the table in FIG. 1B. A cell in state 1110 is stable and remains in state 1110 in the absence of any perturbation, due to the actions of A, B and C on each other and the action of C on D, according to the wiring diagram of FIG. 1A. FIG. 1B shows additional cell state transitions that are imposed by the wiring diagram of FIG. 1A. Together, all the transition pairs of FIG. 1B establish a map of trajectories in the state space (the N-dimensional space that contains all possible gene activity patterns). A cell state (or group of cell states) that progressively transition or ‘drain’ to themselves are, by definition, self-stablizing attractor states (e.g. state 1110 discussed above and shown as in FIG. 1C) and the group of states that ‘drains’ into the same attractor state form the ‘basin of attraction’ of that state. The trajectories, attractors, and their basins of attraction give a structure to the cellular state space. In this example, cell state space is compartmentalized into the 4 basins of attraction: I, II, III, and IV (FIG. 1C).

This 4 gene network illustrates several general features of the invention. In general, a cell state is defined by assigning a vector to a cell, wherein the vector represents the specific pattern of genome-wide activity for that cell in cell state space at a given time; thus, it is a time-dependent state vector. According to the invention, genome-wide cell activities are measured by assaying the activity level of each of a plurality of markers. Preferred markers include gene expression levels, protein expression levels, and protein phosphorylation levels, or any combination thereof. Accordingly, in a method of the invention, each component of a vector is a value assigned to a cell activity marker that is measured. The vector is therefore the set of values of the plurality of markers being studied at a given time. Since each marker is a coordinate of the vector, the dimensionality of the vector space is determined by the number of cell activity markers being studied.

According to the invention, a cell state space can include all patterns of cell-wide activity that are theoretically possible. A preferred cell state space includes only natural patterns of cell-wide activities. Natural patterns of genome-wide cell activities are constrained by the fundamental gene and protein networks of a cell as exemplified above. In a preferred embodiment of the invention, a cell state is represented by a vector in n-dimensional space where n is the number of cell-wide activity markers measured.

Different cell states are characterized by different genome-wide cell activity profiles, for example, by different gene expression profiles. According to the invention, a cell state can be any behavioral state of interest, including primitive undifferentiated cell states, transitional cell states, diseased cell states including metabolic and pathogen-induced diseased states , partially differentiated and terminally differentiated cell states, growing, apoptotic, contractile, or motile states; cell states that result from a response to a perturbation, including addition of drug, toxin, heat, or mechanical force.

Experimental observation of cell dynamics indicate that stable cell states dynamically correspond to “attractor” states of the network of molecular interactions as exemplified above. An “attractor” cell state is a cell state that is maintained even in response to minor perturbations in cell activity (e.g. perturbations in the expression or activity levels of one or a few molecules), whether due to an external perturbation or to a natural intracellular variation in cell activity. Novel experimental work on the regulation of cell fates, i.e. the switch between cellular states, including proliferation, differentiation and commitment to cell death (apoptosis) has revealed that the dynamics of cell states reflects the general dynamics of a regulatory network with attractor states. In fact, transitions between distinct cell states are governed by a network of protein and gene interactions. Thus, cell fates (proliferation, differentiation and apoptosis) are the attractors within the underlying molecular regulatory network. In particular, the proliferation state which consists of recurring gene activity states as the cell undergoes division cycles would correspond to a limit-cycle attractor as shown for attractor state IV in FIG. 1C. Each attractor cell state is located in a basin of attraction on a topographical landscape of cell states. This basin of attraction contains cell states with small differences in cell activity when compared to the attractor cell state and which transition to the attractor cell state as a result of dynamic changes in the pattern of their cellular activity (due to the constraints of the underlying structural, functional, and regulatory interactions between the molecular components that comprise the cell, as illustrated in the 4 gene system discussed above). However, when a cell in a stable cell state is subjected to a large perturbation including multiple molecules, it may transition from one basin of attraction to another basin of attraction. According to the invention, a regulatory switch of cell fate or between different stable behavioral states (e.g. stimulating proliferating cells to enter differentiation) corresponds to a transition between two attractor states in response to such a large (but defined) perturbation and is manifest as a large jump of the state vector in cell state space, followed by smooth (constrained) movement towards the new attractor. Such attractor transitions with approaches along constrained trajectories in cell state space to the new attractor state characterize cellular processes that mediate important state transitions and form a basis of the invention for analyzing genome-wide changes in molecular activities within cells.

In more general terms, any change in biological state or process at the cell, tissue or organism level that results from networked molecular interactions, such as immune activation, inflammation, angiogenesis, malignant transformation and cancer, or response to drugs or toxins correspond to attractor switches or a travel along extended trajectories. Therefore, the model of a network of molecular interactions and the emerging structure of the state space establishes a novel formal framework for analyzing and representing complex biological processes in health and disease which involve a complex network of molecular interactions.

As discussed above, a cell state can be defined by its characteristic genome-wide activity profile. In a preferred embodiment, a cell state is defined by its gene expression profile. In an alternative embodiment, a cell state is defined by its protein expression profile. A cell state may also be defined by a profile of protein activation, for example, by a pattern of protein phosphorylation, subcellular localization, cleavage status, or other posttranslational modification. Finally, a cell state can be defined by any combination of the above profiles and may involve other cellular or extracellular components, such as glycoproteins, RNAs, lipids, carbohydrates, or small chemicals.

Gene and protein expression levels can be measured using arrays of specific nucleic acid probes or antibodies. In the case of genes and proteins, genome-wide monitoring of such activities can be performed using massively parallel approaches such as array-based methods. Preferred embodiments measure the activity of between 1,000 and 30,000 genes or gene products, and preferably between 10,000 and 50,000, and more preferably, the activity of the entire genome. Depending on the cell process considered, the cellular level of hundreds to thousands of relevant metabolic molecules and signaling chemicals, such as glycogen, glucose, cholesterin, phosphatidyl inositides, cyclic nucleotides, calcium, lactate, and other molecules, can be measured and treated as components of the cellular state vector in addition to those resulting from gene or protein arrays.

According to one aspect of the invention, a cell state is represented by assigning a time-dependent vector to a cell, wherein the components of the vector represent the activity levels of the genome-wide activity markers that were assayed at a given time-point. To describe the dynamics of the vector, the value of each activity component aᵢ(t) is normalized relative to a reference point that is typically the initial state prior to induction of a process of interest, aᵢ(t=0).

According to one embodiment of the invention, the activity of various molecules is studied for a chosen biological process. This means that the activity levels are measured as a function of time over the course of the process. An important aspect of the present invention is that it does not require that the molecules (genes, proteins, or other molecules) be known to be associated with the process in question in order to characterize the dynamic features of the process. All that is required is that a genome-wide set of markers can be monitored over the time-course of a defined transition between different cellular functional states. According to the invention, a “process” can be an apparently continuous or abrupt transition from one discrete cell behavioral state to another, or a cellular response to an external perturbation such as exposure to a pathogen, drug, toxin or other compound, or part of a natural or pathological process without any obvious triggering event, such as development, wound healing, angiogenesis, malignant transformation, tumor progression, or any progressive switch from normal to disease or vice versa.

Such a transition of state is illustrated in the simplified model 4 gene system described above. One example of cell state change over time is provided by the transition from attractor state 0000 (molecules A, B, C and D are OFF, illustrated in basin I of FIG. 1C) to attractor state 1110 (gene A, B and C are ON, and D is OFF). This transition is initiated by a perturbation (for example, receptor activation) that switches gene D ON, thereby changing the 0000 cell state to an 0001 cell state, which is in basin II shown in FIG. 1C. As discussed above, the result of this perturbation is that the cell transitions from state 0000 to state 1110 via the following sequence of cell states: 0001 (due to the perturbation), then 1001, followed by 1101, then 1111, and finally 1110 (due to “updating” of the cell state enforced by the underlying wiring diagram as explained above). This series of cell states is an example of a trajectory through cell state space. Accordingly, this trajectory consists of the following “time course” of gene activation patterns (network states) consisting of 6 time points:

0000-0001-1001-1101-1111-1110.

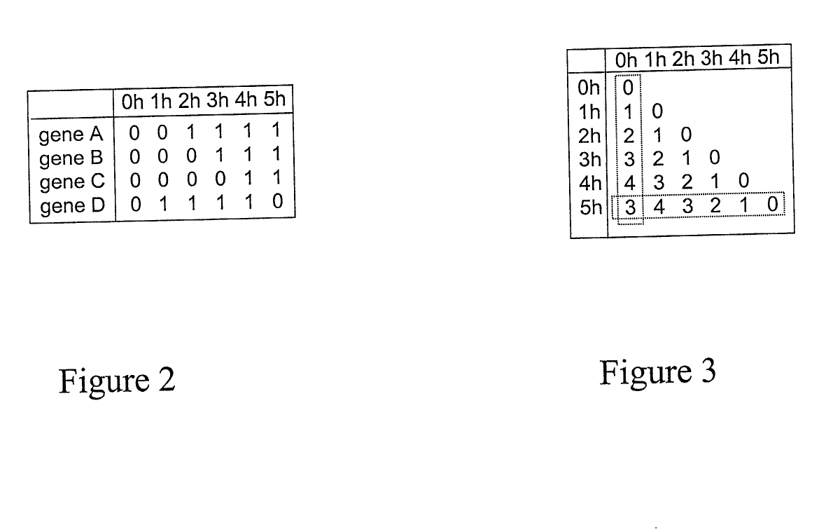

This sequence can be viewed as the displacement of a state vector in the N-dimensional state space whose components are defined by the activity values of the individual molecules. In an example of an experiment where the underlying wiring diagram is not known, the transition from a first cell state (e.g. a first attractor state) to a second cell state (e.g. a second attractor state) is described by a dataset of measured gene activation profiles which is typically presented in a table form of relative expression values measured over the time course of the transition between states. Such a table is illustrated in FIG. 2 for the 4 molecule binary network described above. In this example the gene activation profile of the 4-molecule cell is measured every hour after the perturbation, for 5 hours.

According to a preferred embodiment of the invention, for a cell transition being analyzed, a START or departure point in state space is defined as the first point for which a molecule activity pattern is available (in the 4 molecule example discussed above the start point is 0000). Typically, the START point is based on a measurement carried out before initiation of the transition process. An END or destination point is defined as the last point measured and in practice corresponds to a location within the attractor state if the system is allowed to progress until steady-state is reached (in the 4 molecule example discussed above the end point is 1110). The START and END points are not in the same basin of attraction if the process being considered is triggered by a perturbation that leads to a transition from one attractor state to another attractor state.

According to the invention, during a cellular transition process, molecular activities occur in a characteristic time-dependent pattern that is determined by the functional constraints imposed on the cell by the underlying biological properties of the cell. Indeed, the underlying biological properties of a cell determine the genes and proteins that are activated or inactivated during any given cellular event, and their time-dependent pattern of activation/inactivation. The fact that the patterns are dependent on the underlying biology of the cell means that the general patterns of time-dependent behavior will be reproducible for a given cell in a given environment. Accordingly, a data set of cell state activities measured during the course of a biological event is analyzed to identify a pattern of cell-wide changes characteristic of the cellular transition process.

Preferred cellular processes include transitions from disease to non-disease states, responses to pathogens, drugs, candidate drugs, toxins, temperature, mechanical forces, or any other environmental stimulus. Cellular transition processes also may involve changes referring to cellular aging, cell death, differentiation, growth, motility, contractility, and other characteristic cellular behaviors.

According to the invention, cellular processes may include transitions in cell populations composed of a single cell type as well as concerted transitions in a group of cells composed of different cell types. In preferred embodiments, methods of the invention are used to analyze transition processes in tissues. In all cases, the transition can be spontaneous or in response to an external perturbation. The transition can be between two stable or oscillatory states, or between phenotypically distinct, but non-stationary states. Examples of transitions within a single cell type include switching between distinct cell fates, such as between growth, differentiation, and apoptosis in endothelial cells, functional activation in macrophages, and between replication and senescence in non-transformed cells. Examples of transition processes within mixed cell populations include normal processes, such as wound healing and tissue development. Transition processes also include pathological processes, such as the progress of diseases to recognizable clinical states and malignant transformation, including the switch to the angiogenic state, the switch from non-invasive to invasive growth of tumors, and remission in response to drugs or other therapeutic agents.

According to the invention, a time-dependent analysis involves obtaining genome-wide measurements at multiple time points during the process of transition between distinct cellular behavioral states. The preferred method involves continuous read-out of multiple gene or protein activities. However, effective analysis may be carried out by measuring genome-wide activities at time points preferably spaced every 30 minutes to 6 hours during a transition process. Less preferably, time points may be carried out every 6 to 24 hours. However, in all cases, it is preferred that the time points be spaced regularly throughout the transition process that is being studied. At least 1 time point prior to the initiation of the transition and at least 1 time point after the second cell state is attained and has reached steady-state are preferably included in the analysis.

For each time point, a vector is assigned to the cell state measured as a function of the genome-wide cell activities (e.g. levels of gene or protein expression or function) as discussed above. According to the invention, a time-dependent pattern of cell activities is obtained by analyzing the cell state vectors at each time point during a biological process.

According to methods of the invention, novel mathematical algorithms based on the cell's use of dynamic networks to process biological information are integrated with experimental cell systems that are optimally designed for data acquisition and analysis. In one aspect of the invention, cell state vectors that are obtained for each time point during a cellular transition process are compared. A comparison is performed to produce a mathematical representation of the genome-wide changes that occur during the cellular process. This representation is a dynamic signature of the trajectory of the cell-state vector through cell-state space during the cellular process being studied.

An example of an algorithm according to the invention is illustrated by the discrete 4-molecule model system described above, which is constrained in its activity pattterns based on the underlying wiring network shown in FIG. 1A. However this algorithm can be applied to continuous value expression data from microarray-based monitoring of the transcriptome or proteomic analysis during a variety of biological

$${D}_{ij}^{2} = {\sum }_{g} {( {x}_{g i} - {x}_{g j} )}^{2} / N$$

processes. In this example, a distance matrix is calculated for the distances (“pattern dissimilarity”) between all the possible pairs of measured time-point patterns. A common distance measure is the Square Euclidian distance (D2) between the two patterns i and j:

where g is the index indicating the molecule (A, B, C and D) and N the total number of molecules. For the 4-molecule binary system, the Hamming distance Dₕ, which corresponds to the Euclidian distance without normalization by N, serves as a simple distance measure and is used here, as a non-limiting example. For instance, from FIG. 2, Dₕ(1h-2h)=1, and Dₕ(0h-2h)=2, indicating that the 2h pattern is more distant (“dissimilar”) from the Oh pattern than is the 1h pattern. The Hamming distance is obtained in the following way: the network state at 1h is: 0,0,0,1 (for genes A,B,C,D, see FIG. 2) and the state at 2h is 1,0,0,1. Thus, the Hamming distance Dₕ(1h-2h) is |(1-0)+(0-0)+(0-0)+(1-1)|=1 The Hamming distances between all the pairs formed by the 6 patterns of process 1 is represented by a distance matrix of inter-pattern distances as shown in FIG. 3. Other sophisticated distance measures can be used depending on particular needs. These include: dot products, (squared) Euclidian distance, (non)-linear correlation measures, mutual information, and other methods known to those skilled in the art.

A practical problem in monitoring the dynamics of gene activity profiles during a cellular transition process, e.g. the switch between two attractor states is: how to characterize and represent in a “compact” way the trajectory in the high-dimensional gene activation state space (N=thousands of genes) of such a switching between cell states and the progressive movement towards attractors. In a typical experiment, a time-dependent pattern of gene expression (from DNA arrays) or protein activity is obtained using tens of thousands to over a hundred thousand data points. According to the invention, this high-dimensional information is compressed, by choosing an appropriate phase portrait, into a single picture that represents the displacement of the cell state vector in gene activity state space to determine, for example, if the process under study is a transition into a new attractor.

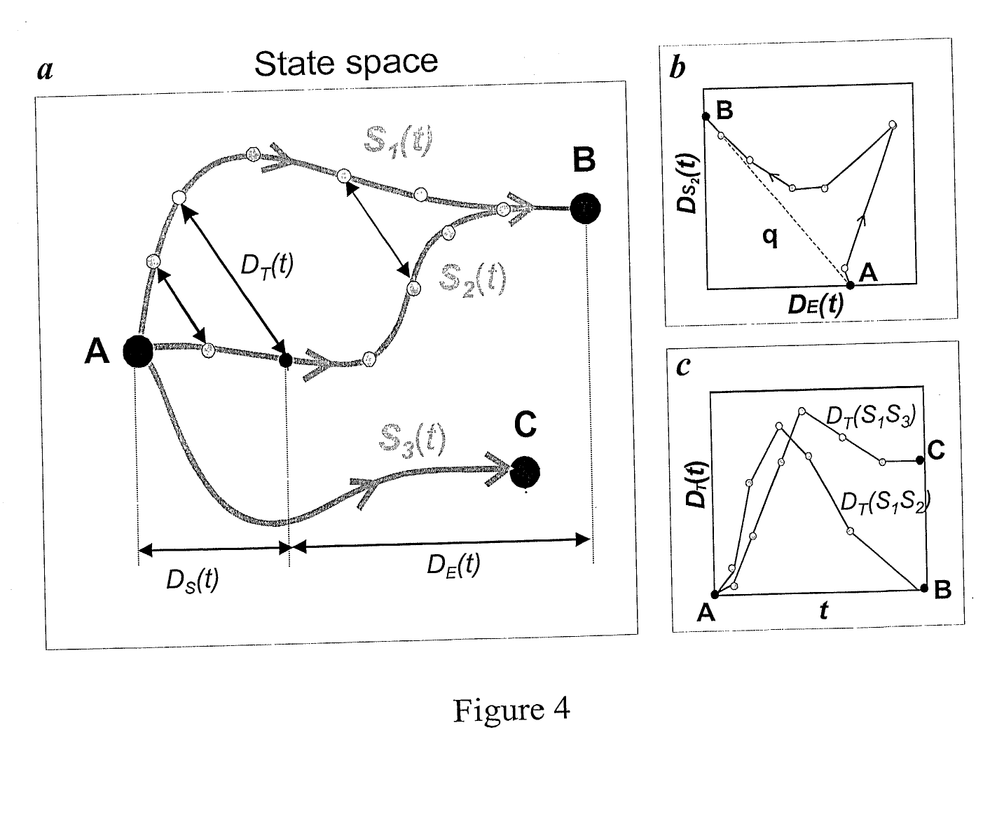

In a preferred embodiment of the invention, a dynamic signature of genome-wide changes during a cellular process is represented graphically as a phase portrait that is characteristic of the cellular transition process. A fundamental finding is that, at least for the class of “well-behaved” networks to which biological regulatory networks belong, the displacement of the network state vector along a trajectory down an attractor basin is on average a relatively smooth process in which the pattern distance D of the network state to the destination state (attractor) at a given time decreases on average monotonically while the pattern distance to the departure state (within the basin) increases on average monotonically. This is not true for dense or chaotic networks. This finding forms an important basis for trajectory representation using phase portraits. It provides one preferred approach for selecting the axis for a 2D phase portrait to represent the state space trajectory of the process, such that the X axis for instance represents the distance of a state at any given time to the destination state (ENDPOINT distance) and the Y axis the distance to the departure state (STARTPOINT distance). FIG. 4a shows a graphical representation of state space trajectories for a theoretical example system in which one departure state (A) and two alternative destination states (B, C) are considered. Thus, for the trajectory A to B, at each time point the cell state is characterized by the STARTPOINT distance D_{S}(t) and the ENDPOINT distance, D_{E}(t). The corresponding phase portrait, exemplifying a transition from one to another attractor, is shown in FIG. 4b.

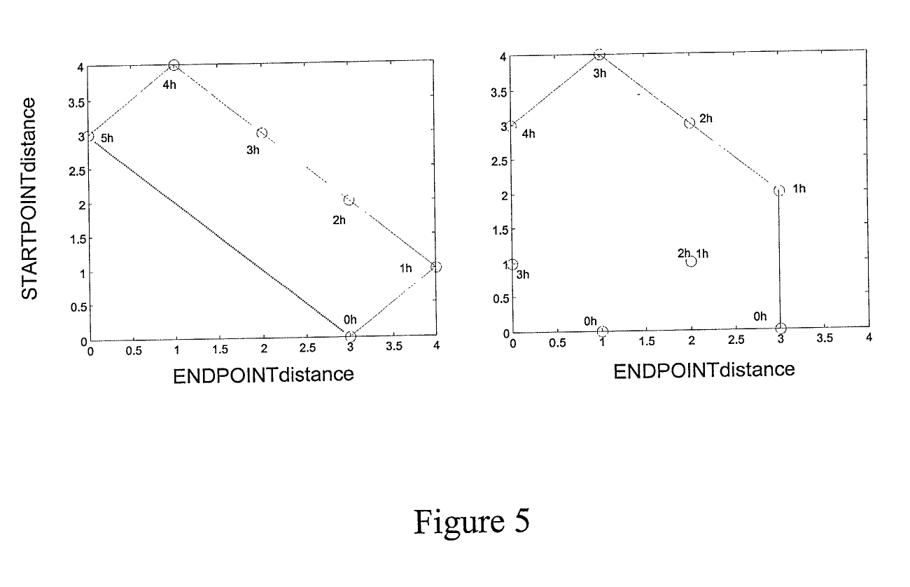

In the 4-molecule example described above, the trajectory of the transition from cell state 0000 to cell state 1110 due to perturbation 0100 (displacement of the state vector in state space) can be depicted as a projection of the state space using appropriate axes that represent absolute or relative distance measures taken from the matrix in FIG. 3. In such phase portraits, ideally the trajectory of a transition to an attractor for a process starting from a state within its basin of attraction, but distant from the attractor, would correspond on average to a straight diagonal line from x=high/y=0 to x=0/y=high (dotted line in FIG. 5). Small deviations of the trajectory from the diagonal represent a specific signature of the process imposed by the wiring of the network and the nature of the perturbation. In contrast, a perturbation from one attractor to another (switch between distinct, stable cell states) exhibits an early, substantial deviation (‘peak’) of the trajectory away from the diagonal towards higher values (the upper right corner of the phase portrait in FIG. 5, left panel). In the example shown in FIG. 5 (left panel), the trajectory indicated by the solid line is bumpy and geometric due to the low number of genes involved. However, since the wiring was chosen to be biologically realistic, most of the trajectory, after initial departure from the diagonal (dotted line) to higher values, moves parallel to that diagonal toward the endpoint (x=0/y=high), indicating the decrease and increase of the respective pattern distances and thus, the movement within an attractor basin, whereas the deviation away from the diagonal indicates that the cell has transitioned between two different attractor basins.

There are other ways to generate phase portraits to characterize a cell event. In principle, any difference between two distance measures from the distance matrix can be used to generate a phase portrait (FIG. 4c). Combined with different ways of generating a distance matrix discussed above, a multitude of such portraits can be generated. The reference points also can be other than the START or END points and, for example, can be the distances to moving points, e.g. D[S(t)−(S(t−1)], which would generate a derivative of the vector displacement. A reference process (for instance a well-characterized process triggered by a known drug or biological perturbation) can be characterized by the vector Sᵣ(t), and the distance of the studied process Sₓ(t), e.g. the response to a novel substance, to the reference process at corresponding time points can be calculated, Dₓᵣ[Sₓ(t)−Sᵣ(t)] and used to compare a multitude of processes. Such temporal evolution of inter-process or inter-trajectory distance is represented in FIG. 4c.

According to the invention, the shape of a specific portion of a phase portrait may be characteristic of a given cellular process or transition between different cellular behavioral states. In a preferred embodiment, a minimal characteristic portion of a phase portrait is identified. An essential element of the method for identifying characteristic parts of phase portraits is the design of experiments, i.e. the choice of the biological processes that are analyzed and represented in phase portraits. Preferably, processes that exhibit ‘convergence’ (i.e., two processes with different START points states that end in similar END points) or ‘divergence’ (i.e., two processes that begin at the same START point and end in two different, defineable, stable behavioral states due to a difference of the inducing mechanism or additional perturbations) will be chosen since they will cover various dimensions of the cell state space and, thus, provide information for mapping out its structure. In particular, the interprocess distance DXY can be displayed, from which the point of convergence of trajectories can be determined. The common stretch of the trajectory which begins at the point of convergence is then a characteristic part of the phase portrait that can have general significance if it is shown to represent a common path shared by other processes that lead to the same attractor. For example, the activity profile defining the point of convergence might represent a characteristic signature of a process that indicates that cell has passed through a critical functional transition and predicts the outcome (i.e., the end point of the trajectory).

The shape of the trajectory in any of the possible phase portrait representations can be instructive as to the nature of a state transition. In the special case where the distances to the END point and the START point are chosen as the axes, as discussed above, a transition between attractor states is indicated by a deviation from the monotonic decay which would be displayed if the cell progressed within a single basin of attraction into its attractor state (in the absence of any perturbation). Specifically, in the case in which the cell did not switch attractor states, the phase portrait would appear as an approximately straight line directly connecting the START point to the END point. In the case where the cell did switch to another attractor, the shape of the phase portrait plotted on the same axes would vary significantly such that the monotonic decay would be lost and an abrupt deviation in the path (e.g., a ‘peak’ or ‘elbow’-shaped deviation in the normally linear plot) is observed. The ratio of the overall distance traveled during the state transition process and the peak deviation distance from the line of monotonic decay to the tip of the “elbow” is related to the relative stringency of the control of the transition by the regulatory network. For instance, a process of switching from a differentiated state to a proliferative state can be compared to the process of the switching from the proliferative to the differentiated state. In this particular case, it can be shown that the latter is less stringently controlled, i.e. has a higher probability to occur given a randomly chosen perturbation. This is in accordance with physiological epigenetic barriers that restrict the entry of differentiated cells into the proliferative state.

Such measures of stringency of state transition control imposed by the cell's molecular regulatory network are important for the assessment of developmental potentials (maturation, terminal differentiation, transdifferentiation, retrodifferentiation) of stem cells or cancer cells that correspond to immature stages of cell differentiation. In a more encompassing view, knowledge of the stringency of transitions represents a tool for quantifying the rule-like behavior of biological processes and an opportunity for therapeutic interference.

Characteristic signature profiles or phase portraits of the invention can be used as discussed in the following sections. The invention represents a major departure from conventional data-mining technology, because it directly links genomic and proteomic data to the manner in which cells actually process information and thus, permits identification of functionally-linked and mechanistically-relevant groups of genes, proteins and signaling molecules. The invention facilitates the use of cellular activity information by providing it in a form that is functionally relevant and readily exploitable.

According to the invention, a dynamic signature or phase portrait can be obtained for a particular transition in cellular behavior. Because of the constraints of the dynamics and the limited number of trajectories, a dynamic signature or phase portrait is preferably characteristic, and most preferably uniquely characteristic of the cellular transition. In one embodiment of the invention, the dynamic signature or phase portrait is used as a reference to predict the outcome of an experimental cell system based on a comparison of the observed patterns of cellular change in the experimental system with the known patterns of cell change in the reference. In a preferred embodiment, the reference is based on a pattern of cell change that occurs early in the cellular transition, during or just after the deviation from the line of theoretical monotonic decay into the END point state from the START state. Accordingly, early changes observed in an experimental cell system can be compared to the reference in order to ascertain whether the experimental system will follow the same transition as the reference. Such a prediction is most useful in situations where it is advantageous to know the outcome of a cellular system (e.g., cultured cells, tissue sample) in advance. For example, cell fate prediction is useful in disease diagnosis or prognosis, such as cancer diagnosis or prognosis, or when assaying candidate drugs in a drug screening or lead drug validation program. Such cell fate prediction can also be used to predict cell fate in heterogeneous groups of cells such as in a tissue.

A simplified example of a phase portrait comparison is provided by the 4-molecule network described above. The following discussion illustrates how the differences between different cell state transitions are manifested as differences in the 2D phase portrait representations of the cell state trajectories in this example. The transition discussed above (transition 1) was from cell state 0000 to cell state 1110 in response to a perturbation to cell state 0100. The following illustration is based on two additional cell state transitions (transition 2 and transition 3) and the associated time courses of gene activity pattern changes. Transition 2 involves the following sequence of cell states imposed by the wiring network in response to a perturbation that initiates the transition by changing cell state 0000 to cell state 0101: 0000-0101-1101-1111-1110. This transition ends up in the same attractor state (1110) as the transition initiated by the perturbation from state 0000 to state 0100 described above. Transition 3 involves the following sequence of cell states imposed by the wiring network in response to a perturbation that initiates the transition by changing cell state 0000 to cell state 0010: 0000-0010-1000-0100. This transition ends up in attractor state (0100) as shown by a dotted line in FIG. 5 (right panel).

Transitions 2 and 3 both represent cell transitions starting from the attractor state 0000. Transition 2 has a trajectory that converges with the trajectory of transition 1 as shown by the phase portraits and ends in the same attractor state, but transition 2 starts differently and is shorter by one time step. In contrast, the trajectory of transition 3 ends in another attractor state. Subjecting these transitions to the same type of analysis illustrates how the phase portrait representation reveals similarities and differences in the transitions that are due to the use of different regions of the state space (FIG. 5). It should be noted that in this highly simplified example, the 4 binary molecule system with short trajectories gives rise to random, erratic behavior that has a substantial impact on the overall output. Nevertheless, FIG. 5 (right panel) shows that the phase portrait representation of the trajectory of transition 2 clearly resembles the shape of transition 1 shown in FIG. 5 (left panel), whereas the phase portrait representation of the trajectory of transition 3 (FIG. 5, right panel) which switches between different attractor states deviates significantly from that of transition 1.

To compare trajectories, the phase portraits of trajectories of various processes as defined by genome-wide molecular activities or a selected subset of activities can be subjected to conventional cluster analysis or classification approaches (e.g., conventional pattern recognition algorithms, genetic algorithms, or neural network-based algorithms) known to those skilled in the art.

According to the invention, dynamic signatures and phase portraits are useful to identify important genes underlying a transition from one cell state to another cell state. In one aspect of the invention, the information in the dynamic signature can be used to identify one or more genes (or other relevant molecular activities) that are effectively barriers to the transition, such that the transition will not occur unless the activities of those genes or proteins are changed, but once these molecular activities are altered, the transition runs to completion. For example, in a phase portrait representing cellular activity changes relative to a start point and an end point of a transition, an important gene or set of genes may be identified as one that is responsible for a significant deviation from the direct (monotonic) trajectory from the start point to the end point. These genes (or molecules) may be identified by progressively subtracting genes or subsets of genes from the set of genes used to calculate the phase portrait until the minimal set of genes necessary to produce the characteristic deviation from monotonic trajectory (e.g., the elbow form in the portrait) is identified. Alternatively, different sets of genes may be progressively clustered together to carry out this form of analysis. Both approaches may be accomplished using standard clustering and iteration techniques available to those skilled in the art of computer science, engineering and bioinformatics. In this manner, it should be possible to narrow the number of genes down to a subset that jointly contribute maximally to promoting the deviation from the direct START point to the END point trajectory. This set of genes will contain the most likely candidates (individually or as a group) for genes that are causative in the cell state transition process. Such an analysis will significantly increase the accuracy, as compared to conventional bioinformatics and data-mining approaches to (1) sort out “innocent bystander genes” and (2) to identify short acting genes/proteins that act like toggle switches by being active or expressed only during early phases in the transition process.

Accordingly, application of the dynamic network analysis described above to experimental systems that involve transition processes, (e.g. switching between different cell fates) can identify important molecules that have the generic function of triggering cell state transitions (i.e., overcoming the dynamic constraints that establish attractor robustness) without directing the particular path or the final attractor state (cell behavior state). Under the action of this type of molecule, the final cell state will be specified by the action of additional genes/proteins within the cell's regulatory network. An example of this type of a molecule would be ras, which can trigger either proliferation, differentiation, senescence or apoptosis depending on the presence of other cellular activities.

In one embodiment, trajectory phase portraits are constructed by considering only a subset of the genes in a cell. By iterating the process with different subsets of genes, clusters of patterns of dynamic signatures and genes arise. One application is to use these clusters of genes to find those that are most associated with those clusters of trajectories whose phase portrait shape is most indicative of an attractor transition in order to identify those genes/proteins that are likely to be causally involved in triggering the transition. This approach may be used to identify specific molecules that represent new drug targets or potential mediators of toxicity and pathogenicity. In a more general application, this clustering approach, which clusters genes with regard to their effect on the shape of the trajectory phase portrait (including those not involved in state transition) represents an alternative to conventional cluster analysis methods for subcategorizing genes. In methods of the invention, clustering is based on an integrative parameter such as the class of trajectory in state space (e.g. the shape of the phase portrait) that they contribute to. By using an identified distinguishing subset of genes for a trajectory analysis, noise from genome-wide data can be reduced and a finer discrimination between trajectories can be achieved, thereby providing higher resolution analysis.

Every biological process gives rise to a characteristic trajectory given an appropriate choice of the axis dimensions for the phase portraits and a defined subset of molecules considered. The trajectory representation in phase portraits based on the displacement of the state vector in state space over time allows the extraction of a low-dimensional characteristic signature, because each phase portrait is preferably generated in a space of lower dimension than the crude cell activity data. Accordingly, the phase portrait information can be subjected to pattern recognition to identify groups of trajectories that lead to the same attractor states. According to the invention, an analysis of simulated networks shows that many different cell transitions (elicited by different perturbations, i.e. starting from varying initial conditions) that have trajectories leading to the same attractor state exhibit striking similarities near the attractor state.

Methods of the invention are useful to identify a candidate gene for drug screening or for identifying a toxin or pathogen. As discussed herein, algorithms of the invention are useful to identify molecules that are important to cellular transitions and are involved in the early stages of cellular transitions.

According to one aspect of the invention, a candidate molecule for a drug target is a molecule that is causally involved in a disease process. In one aspect of the invention, a candidate gene is involved in the transition from a healthy cell state to a diseased cell state. A useful drug is one that interferes with this molecule and inhibits the transition from a healthy to a diseased state. In another aspect of the invention, a candidate target molecule is involved in the transition from a diseased cell state to a healthy cell state. A useful drug is one that activates this gene or its gene product and promotes transition from a diseased to a healthy state. In particularly preferred embodiments of the invention, a candidate gene for a drug screen is a gene that is involved in the early stages of a transition from or to a diseased cell state.

Methods of the invention are also useful to validate candidate drugs for advancement into animal studies and human clinical trials. As discussed above, methods of the invention are useful to identify targets for drug screens. However, once a target is chosen, methods of the invention are also useful in drug screening and validation assays. Instead of following the potential therapeutic effects of a candidate drug over extended periods of time at the level of functional phenotype, the invention provides reference dynamic signatures that are predictive for a phenotypic outcome and thus can be used to evaluate the effectiveness of drug candidates early during a screening assay. For example, a drug candidate that induces a pattern of cell activities characteristic of a transition from a diseased cell state to a healthy cell state, and preferably characteristic of the early stages of the transition, is chosen for further analysis.

In another aspect of the invention, once a candidate drug is chosen, methods of the invention are also useful to evaluate the drug for toxicity and other, in particular, delayed side-effects. Again, instead of following the toxicity of side effects of a candidate drug over extended periods of time at the functional level, the invention provides a reference (predictive) dynamic signature that can be used to evaluate the properties of a candidate drug early on during an assay by measuring just a set of molecular markers.

In one aspect of the invention, large scale screening of a candidate drugs' effectiveness or toxicity/side effects is performed in model cell systems for which reference dynamic signatures or phase portraits are available. Accordingly, the properties of the candidate drugs can be assessed using software programs to compare patterns of cell activity observed in response to application of a candidate drug with a database of known cell activity profiles extracted from reference trajectory signatures of standard cell transitions (e.g. into a stress response state attractor). Therefore, lengthy animal testing is not required for all the drug candidates and the cost of the drug development process is greatly reduced. Optionally, once a subset of promising drug candidates is chosen using a computer analysis according to the invention, the effectiveness and toxicity/side effects of these candidates may be verified in animal or human clinical trials.

Methods of the invention are also useful to identify dynamic signatures and phase portraits characteristic of cellular toxicity. This information can be used to model a cellular response to a toxic compound. This information is also useful to evaluate the potential toxic effects of a candidate therapeutic compound. In one embodiment, one or more genes or gene products involved in toxicity are also identified. According to the invention, model systems for identifying one or more dynamic signatures characteristic of toxicity include induced liver toxicity, autoimmunity, neurotoxicity, or nepthrotoxicity, and recovery from these toxic states.

In one aspect of the invention, data obtained from the analysis of cell transitions is stored in a computer. The data for each cell transition may be stored as raw data (uncompressed trajectory of cell state vector), or as a dynamic signature in a given, annotated projection (phase portrait) representing changes in cell activity during the cell transition. The data is preferably organized to be accessed and retrieved by a software program that compares known dynamic signatures or phase portraits with experimental or test data. In one embodiment, each dynamic signature is assigned an identifier and stored in a relational database with direct link to the underlying raw data and exhaustive annotation regarding the biological parameters of the cell transition process represented by that given dynamic signature.

In one embodiment of the invention, the data is available on a website. Accordingly, an investigator may access the data to use as a reference to compare to experimental data obtained by the investigator. In a preferred embodiment, the website also provides one or more links to software for use with the data. In another embodiment of the invention, an investigator submits experimental data to a service for comparison with reference information, and the service provides an analysis of the experimental data to the investigator.

### EXAMPLES

The following examples provide further details of methods according to the invention. For purposes of exemplification, the following examples provide details of specific cell types and specific algorithms. Accordingly, while exemplified in the following manner, the invention is not so limited and the skilled artisan will appreciate its wide range of application upon consideration thereof.

### Example 1

### Using a Human Stem Cell System for Data Analysis

Methods of the invention can be applied to any biological system that exhibits a stable switch in cellular behavior or phenotype, whether normal or pathological. Given the value of understanding the genetic basis of the switch between different hematopoietic stem cell lineages, a well-characterized human pluripotent precursor cell line—the HL 60 promyeloid leukemia cell line—provides a useful model system. HL 60 precursors cells can be induced to switch to granulocytes, monocytes, or macrophages based on alterations in experimental conditions. One aspect of the invention is identifying precise conditions necessary to consistently induce the shifts between different cell fates (stable behavior states), and hence different attractor states. An important aspect of this model system is establishing the relationship between individual differentiation paths which exhibit convergence, divergence, and reversibility. Established molecular methods (e.g., immunocytochemistry, FACS cell sorting, SDS-PAGE, Western blots, Northern blots, or other biochemical or molecular biological techniques) can be used to identify cells that have switched, between different phenotypes. These methods can be used to identify experimental conditions necessary to optimally acquire the baseline data for analysis according to methods of the invention, including information on the dose-response for the transition between different cell states and the time required for a stimulated cell to undergo the transition and reach a new steady state. Additional insight into the molecules involved in cell regulation may be gained by carrying out analysis of cells under various experimental conditions that induce forward and reverse transitions between the same two states by adding or removing a common stimulus as well as by analysis of convergent transition processes that involve switching to a common cell state using different stimuli and divergent transition process that involve switching from one state to two different attractor states using different perturbations. Moreover, properties of the attractor can be revealed by using a range of doses or strengths of the stimulator, including “subthreshold doses”. The latter would lead to a perturbation of the attractor state to various degrees with subsequent relaxation back to the same attractor along distinct trajectories.

Cell state transitions that may be studied and characterized in the HL60 model system include: A) induction of a transition from HL60 precursor to a granulocyteby treatment with DMSO; B) induction of a reverse transition from granulocyte to HL60 by removing DMSO; C) induction of a transition from HL60 precursor to granulocyte using all-trans-retinoid acid; D) induction of a reverse transition from granulocytes to HL60 precursors by removing all-trans-retinoid acid; E) induction of a transition from HL60 precursors to a monocyte(s) by addition of NaButyrate; and F) induction of a transition from HL60 precursors to a macrophage(s) by addition of TPA. This approach was used to analyze two convergent processes of HL-60 cell transition from precursor to neutrophil states after stimulation with DMSO and retinoic acid (transitions A and C, respectively). In this experiment, only a relatively low number of genes (<300) were analyzed over 7 time points.

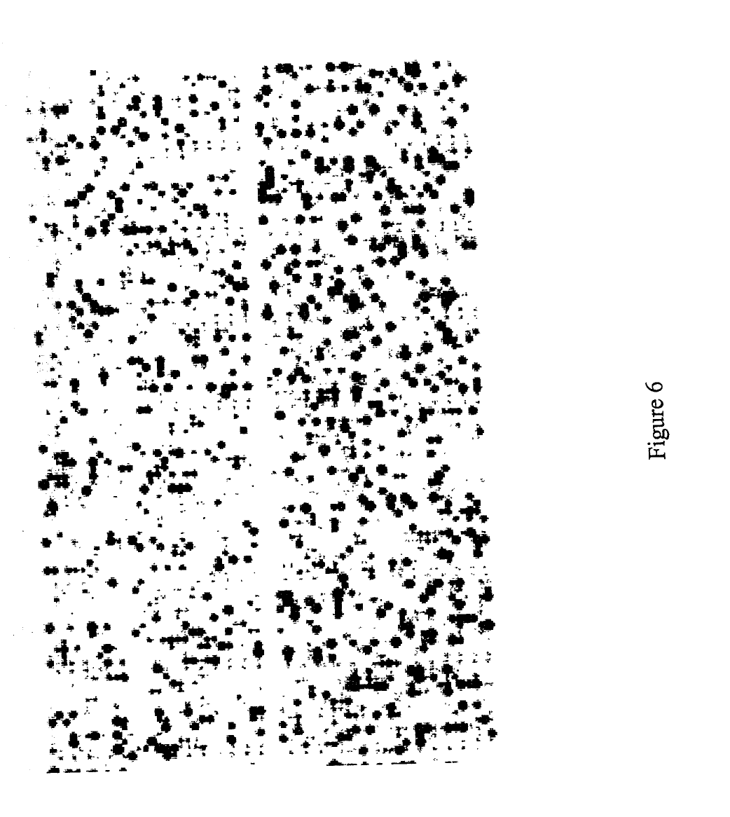

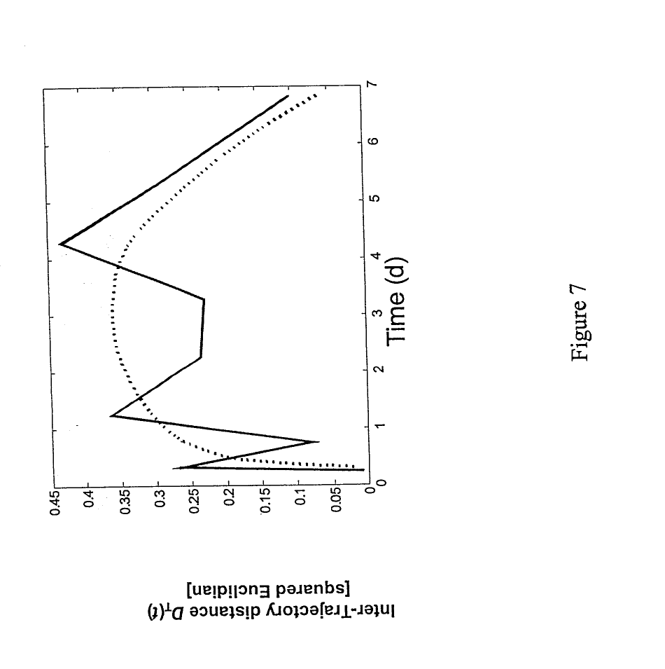

HL-60 cells (1.5×10⁶ cells/ml) were stimulated in parallel cultures to differentiate into neutrophils along two different but convergent paths by treatment with DMSO (S1 path) or retinoic acid (S2 path). At each time point, RNA was harvested from both cultures using RNeasy extraction kit (Qiagen) and subjected to gene expression profiling using Resgen microarray filters from Research Genetics/Invitrogen (the gene filter contains 5000 human cDNAs that were pre-spotted on a 5×7 cm nylon membrane and then hybridized with radioactively labeled cDNA made from total cellular RNA). FIG. 6 shows an example of a filter at one time point showing different expression levels of different genes. Expression levels for each gene were normalized to the 0 time point level for that gene (untreated reference). Genes with expression levels below a 4 fold change relative to the 0 reference were excluded from the analysis. The remaining 281 genes were further analyzed based on the data for the different time points shown in Table 1. In this analysis, an inter-trajectory distance representation was used to compare different trajectories with the cell state space and to identify common paths in cell state transition processes. For each time point, a state vector of length 281 genes was defined for each of the two processes S1(t) and S2(t). The squared Euclidean distance between each pair of state vectors at each time point t was calculated to represent the inter-trajectory distance D_{T}(t)+E[S1(t), S2(t)], where E[x,y] denotes the squared Euclidean distance between vectors x and y. The experimental values are shown as a solid line in FIG. 7 and progressively increase from the zero to larger inter-trajectory distances as the paths temporarily diverge. The values then decrease towards a zero distance value as the two different trajectories converge on the same differentiation state. The experimental values are consistent with a bell-shape curve (shown as a dashed line on FIG. 7) that would be expected for global behavior associated with a process with initial divergence (due to different stimuli) and subsequent convergence (due to a common attractor).

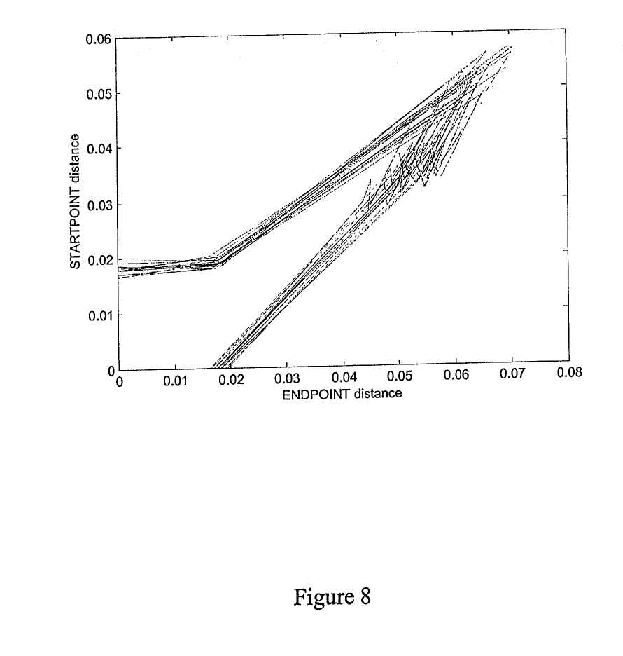

Table 1 shows data for the 281 genes for a retinoic acid induced switch from a proliferation state to a differentiation state was also used to generate phase portraits (using methods described above, discuss in more detail). To show the robustness of the phase portrait, 80% of the genes were randomly selected for several phase portrait calculations. The procedure was repeated 10 times for 10 different sets and the 10 phase portraits were overlaid as shown in FIG. 8. The common shape of the elbow confirms the existence of an attractor switch and that the phase portrait is representative of the underlying cellular information processing network, rather than being limited to a particular form of analysis.

TABLE 1

| | Norm | | | | | | |
|---|---|---|---|---|---|---|---|
| Gene Identifier | T = 0 | 2 hours | Day 2 | Day 3 | Day 4 | Day 5 | Day 7 |
| AA419177 | 1 | 0.875436 | 0.835489 | 0.922322 | 0.7491258 | 1.031471 | 0.943995 |
| AA425299 | 1 | 0.787678 | 0.815704 | 0.925529 | 0.8204677 | 1.195517 | 1.004857 |
| AA425900 | 1 | 0.881076 | 0.824078 | 0.830613 | 0.8802896 | 0.960352 | 0.889591 |
| AA425934 | 1 | 0.942945 | 0.870432 | 0.955803 | 0.8064624 | 0.82408 | 0.839574 |
| AA427735 | 1 | 0.798072 | 0.856427 | 0.93547 | 0.8230401 | 1.161943 | 0.914374 |
| AA427782 | 1 | 0.96214 | 0.914671 | 0.873802 | 0.8728433 | 1.025765 | 1.151935 |
| AA427899 | 1 | 0.883142 | 0.82705 | 0.904024 | 0.8096426 | 1.157108 | 0.924558 |
| AA430667 | 1 | 0.782741 | 0.753333 | 0.942541 | 0.7266643 | 1.153977 | 1.962214 |
| AA431206 | 1 | 0.910988 | 0.854816 | 1.076444 | 0.798226 | 0.950312 | 0.890049 |
| AA431430 | 1 | 1.069634 | 1.06855 | 0.970946 | 1.0944194 | 0.964547 | 1.02102 |
| AA431438 | 1 | 1.437604 | 1.457693 | 1.399982 | 1.5943736 | 1.084145 | 1.01214 |
| AA432248 | 1 | 1.010142 | 1.116602 | 1.088907 | 1.0979784 | 0.94573 | 1.041075 |
| AA432270 | 1 | 0.892752 | 0.881166 | 0.819044 | 0.8426344 | 0.882833 | 0.920451 |
| AA434390 | 1 | 1.590329 | 1.519114 | 1.323171 | 1.5357458 | 0.813882 | 1.058377 |
| AA434404 | 1 | 0.744441 | 0.798041 | 0.90502 | 0.734297 | 1.180243 | 0.977629 |
| AA434411 | 1 | 1.330095 | 1.409027 | 1.244667 | 1.4240387 | 1.19774 | 1.156291 |
| AA436187 | 1 | 1.026702 | 1.15748 | 1.148357 | 1.2743599 | 1.317578 | 1.093477 |
| AA437226 | 1 | 0.891303 | 0.979977 | 1.006299 | 0.9809394 | 1.055347 | 0.918927 |
| AA443570 | 1 | 1.183688 | 1.203509 | 1.030571 | 1.2354619 | 0.850006 | 1.054914 |
| AA443624 | 1 | 0.99148 | 1.058306 | 1.010386 | 0.9796124 | 0.944822 | 1.091466 |
| AA447995 | 1 | 1.254831 | 1.146012 | 1.197204 | 1.0729722 | 0.909231 | 0.865739 |
| AA448001 | 1 | 0.796025 | 0.788625 | 0.739974 | 0.777407 | 0.993951 | 0.842607 |
| AA448271 | 1 | 0.92369 | 0.931195 | 0.999862 | 0.9187359 | 0.858502 | 0.896416 |
| AA453289 | 1 | 0.971444 | 1.023694 | 0.916356 | 0.8977086 | 0.99226 | 1.158241 |
| AA453458 | 1 | 0.859894 | 0.796335 | 0.912086 | 0.763358 | 1.030879 | 0.83118 |
| AA453520 | 1 | 1.047549 | 1.05178 | 1.111148 | 1.1360994 | 0.912595 | 0.863601 |
| AA453579 | 1 | 0.982642 | 0.917661 | 0.823662 | 1.886906 | 0.912221 | 0.851654 |
| AA453618 | 1 | 0.883191 | 0.877862 | 0.850503 | 0.8526188 | 0.748798 | 0.807211 |
| AA454218 | 1 | 0.833967 | 0.899746 | 1.18295 | 0.7678 | 0.953951 | 0.966263 |
| AA454228 | 1 | 0.823051 | 0.832502 | 0.873428 | 0.7491592 | 1.116492 | 1.17462 |
| AA454597 | 1 | 1.323816 | 1.278709 | 1.330173 | 1.1535578 | 1.944533 | 0.930813 |
| AA454756 | 1 | 0.966541 | 0.991426 | 0.92038 | 0.9483785 | 0.835349 | 0.878302 |
| AA454840 | 1 | 0.890218 | 0.885807 | 0.873876 | 0.7985587 | 1.115799 | 1.127461 |
| AA454864 | 1 | 0.777257 | 0.764973 | 0.787858 | 0.7318329 | 0.906024 | 0.878217 |
| AA454867 | 1 | 0.92745 | 0.91857 | 0.880565 | 0.9605639 | 1.008406 | 1.353231 |
| AA455111 | 1 | 1.895893 | 1.994404 | 1.765722 | 2.0494718 | 0.888814 | 0.886826 |
| AA455267 | 1 | 1.233779 | 1.095462 | 1.057659 | 1.0880752 | 0.963535 | 1.078465 |
| AA455291 | 1 | 0.897535 | 0.877632 | 0.863552 | 0.9771493 | 1.018693 | 1.866667 |
| AA456595 | 1 | 1.267163 | 1.212378 | 1.109312 | 1.2598922 | 0.734296 | 0.911706 |
| AA457117 | 1 | 0.796605 | 0.812427 | 0.972437 | 0.6514949 | 0.977905 | 1.027708 |
| AA457153 | 1 | 0.926376 | 0.854564 | 0.859946 | 0.7510533 | 0.959673 | 0.976587 |
| AA457155 | 1 | 1.769834 | 0.733564 | 0.751479 | 0.6520234 | 1.011598 | 0.87398 |
| AA458460 | 1 | 0.857557 | 0.833628 | 0.810541 | 0.8123158 | 0.854427 | 0.874542 |
| AA458471 | 1 | 0.854429 | 0.866288 | 0.778599 | 0.8271324 | 0.923868 | 0.789103 |
| AA458480 | 1 | 1.072973 | 1.048641 | 0.935305 | 1.1339863 | 0.730525 | 0.753863 |
| AA458882 | 1 | 0.925959 | 0.934591 | 0.954377 | 0.8783145 | 0.923573 | 0.885009 |
| AA458959 | 1 | 0.819279 | 0.832996 | 0.827597 | 0.7539867 | 1.160124 | 1.37078 |
| AA459381 | 1 | 1.449469 | 1.403271 | 1.588089 | 1.515739 | 0.924835 | 1.038417 |
| AA459390 | 1 | 1.116642 | 1.130292 | 0.925377 | 1.027909 | 1.930072 | 1.268517 |
| AA459697 | 1 | 0.837988 | 0.826048 | 0.828838 | 0.7473799 | 1.018472 | 1.262136 |
| AA460295 | 1 | 1.551135 | 1.573693 | 1.274686 | 1.5003254 | 0.848991 | 1.947473 |
| AA461301 | 1 | 0.960003 | 0.969952 | 0.955011 | 0.971425 | 0.954626 | 0.926051 |
| AA460313 | 1 | 0.847252 | 0.850349 | 0.84908 | 0.8606085 | 0.817306 | 0.834553 |
| AA460950 | 1 | 1.196291 | 1.233952 | 1.123533 | 1.4448756 | 0.964367 | 0.983686 |
| AA461304 | 1 | 0.733743 | 0.838424 | 0.635215 | 0.8389409 | 1.108695 | 0.969135 |
| AA461497 | 1 | 1.133773 | 1.271399 | 1.010237 | 1.3815923 | 0.890071 | 1.233597 |
| AA463924 | 1 | 0.861602 | 0.831462 | 0.740059 | 0.8508202 | 0.869023 | 0.812549 |
| AA463972 | 1 | 0.795508 | 0.773222 | 0.684787 | 0.7533122 | 0.992212 | 0.88446 |
| AA464195 | 1 | 1.380221 | 1.488049 | 1.041388 | 1.4291506 | 0.845389 | 1.089885 |
| AA464542 | 1 | 1.032146 | 1.100046 | 1.006327 | 1.1798979 | 0.98679 | 1.140443 |
| AA464568 | 1 | 0.737662 | 0.757539 | 0.71912 | 0.7137277 | 1.138787 | 0.987281 |
| AA464704 | 1 | 0.943269 | 0.9748 | 0.94161 | 0.902044 | 0.960711 | 1.367244 |
| AA464741 | 1 | 1.387819 | 1.413542 | 1.114503 | 1.5225363 | 0.851714 | 1.205585 |
| AA477428 | 1 | 1.024914 | 0.958903 | 0.996385 | 0.8428914 | 0.86759 | 0.89182 |
| AA478273 | 1 | 1.083672 | 0.98188 | 1.231119 | 1.0825446 | 0.978846 | 1.11617 |
| AA479888 | 1 | 1.075639 | 0.953505 | 1.020391 | 0.9564209 | 0.781798 | 0.875476 |
| AA481464 | 1 | 1.208396 | 1.314756 | 1.1753334 | 1.3618307 | 0.852931 | 0.90396 |
| AA485365 | 1 | 0.757145 | 0.725206 | 0.826947 | 0.6815137 | 1.023698 | 0.947751 |
| AA488084 | 1 | 0.882011 | 0.870648 | 1.033359 | 0.8067969 | 1.100393 | 0.952415 |
| AA491302 | 1 | 0.892332 | 0.911169 | 0.872702 | 0.8887585 | 1.000222 | 1.316557 |
| AA496357 | 1 | 1.315865 | 1.316468 | 1.330016 | 1.533509 | 0.926734 | 0.930198 |
| AA504465 | 1 | 1.1828 | 1.381174 | 1.428364 | 1.309482 | 0.88694 | 1.871708 |
| AA608988 | 1 | 0.825056 | 0.831953 | 0.826395 | 0.9054905 | 1.253743 | 1.058937 |
| AA609609 | 1 | 0.888967 | 0.946087 | 0.943185 | 0.9576037 | 1.043821 | 0.979563 |
| AA609655 | 1 | 0.963865 | 1.0602 | 1.108154 | 1.0479066 | 1.428309 | 1.154394 |
| AA609976 | 1 | 1.175435 | 1.027531 | 1.15704 | 1.1111383 | 0.950385 | 1.06626 |
| AA620859 | 1 | 0.796231 | 0.737529 | 0.879024 | 0.8639905 | 1.026068 | 0.9873663 |
| AA625806 | 1 | 1.308021 | 1.279148 | 1.201891 | 1.3120814 | 0.80652 | 0.88441 |
| AA629958 | 1 | 1.643235 | 1.544398 | 1.332154 | 1.670239 | 0.99312 | 1.044238 |
| AA629838 | 1 | 0.833935 | 0.893918 | 0.916181 | 0.9815549 | 1.126303 | 1.043912 |
| AA629862 | 1 | 0.994282 | 10.96572 | 1.14485 | 1.0177077 | 0.780015 | 0.90318 |
| AA629923 | 1 | 0.985564 | 0.869549 | 0.981733 | 0.8253315 | 0.968491 | 0.936302 |
| AA630104 | 1 | 0.970049 | 0.97338 | 1.026699 | 1.1583077 | 0.954968 | 0.980904 |
| AA630776 | 1 | 0.740138 | 0.794227 | 0.89374 | 0.8153598 | 1.16854 | 1.003536 |
| AA633811 | 1 | 1.030489 | 1.061643 | 1.251714 | 1.0725573 | 0.937001 | .090414 |
| AA644448 | 1 | 1.078733 | 1.159547 | 0.789494 | 1.2210384 | 1.024736 | 1.041484 |
| AA644657 | 1 | 0.935039 | 0.907387 | 1.006091 | 0.9473 | 1.228312 | 1.0654 |
| AA669055 | 1 | 0.930566 | 1.031091 | 1.047878 | 1.0425358 | 1.434655 | 1.34878 |
| AA669443 | 1 | 0.824647 | 0.843295 | 0.866203 | 1.0000285 | 1.059464 | 0.992772 |
| AA670347 | 1 | 0.905755 | 0.950636 | 1.0276 | 0.8973778 | 0.829446 | 0.895399 |
| AA670382 | 1 | 1.050798 | 1.023771 | 1.080663 | 1.0625352 | 0.841556 | 1.052782 |
| AA679345 | 1 | 0.92854 | 0.974713 | 0.989329 | 1.0150822 | 1.042896 | 0.893881 |
| AA682851 | 1 | 0.909282 | 0.894382 | 1.032386 | 0.8884252 | 1.352655 | 1.133008 |
| AA699469 | 1 | 1.075728 | 1.021392 | 1.271303 | 1.0885083 | 1.274186 | 1.272749 |
| AA699560 | 1 | 0.906072 | 0.894576 | 1.11401 | 0.8542067 | 0.93751 | 0.905466 |
| AA699926 | 1 | 0.88167 | 0.849501 | 0.913918 | 0.8561704 | 0.978978 | 0.962523 |
| AA700322 | 1 | 1.02859 | 1.144722 | 1.159003 | 1.2756674 | 0.917037 | 0.853805 |
| AA702541 | 1 | 1.142777 | 1.307329 | 1.197854 | 1.1329165 | 0.951556 | 1.033815 |
| AA702544 | 1 | 1.115174 | 1.216008 | 1.213892 | 1.1495604 | 0.889691 | 0.854449 |
| H10939 | 1 | 0.941262 | 1.020764 | 1.129141 | 0.9321416 | 1.135937 | 0.940223 |
| H24316 | 1 | 1.007981 | 0.867122 | 1.045331 | 0.9800246 | 0.918559 | 0.913112 |
| H27864 | 1 | 0.906484 | 0.951529 | 1.084217 | 0.8645424 | 0.759026 | 0.79802 |
| H29290 | 1 | 1.071826 | 1.24612 | 1.098582 | 1.0620892 | 1.016214 | 0.942242 |
| H42894 | 1 | 0.96352 | 0.98971 | 0.843188 | 1.0947434 | 0.977232 | 0.968708 |
| H53073 | 1 | 0.984873 | 0.9905 | 0.89802 | 1.0042414 | 0.997413 | 0.932498 |
| H53703 | 1 | 1.234358 | 1.229073 | 1.484737 | 1.4104734 | 1.045139 | 1.17111 |
| H56029 | 1 | 0.992728 | 1.02315 | 0.946887 | 1.1107569 | 1.429698 | 1.419934 |
| H57136 | 1 | 1.01172 | 0.955625 | 1.00712 | 1.1010246 | 1.000733 | 0.944374 |
| H90894 | 1 | 1.068359 | 0.776166 | 0.950109 | 0.8843702 | 0.951051 | 1.081895 |
| H93393 | 1 | 1.235111 | 1.189474 | 1.156721 | 1.2733866 | 1.00233 | 1.012214 |
| H98134 | 1 | 0.919026 | 0.953002 | 1.15893 | 0.8774697 | 1.116585 | 1.07793 |
| H98201 | 1 | 1.059483 | 0.902025 | 0.877147 | 0.8503653 | 0.892558 | 0.981813 |
| N21407 | 1 | 0.96635 | 1.055713 | 1.069654 | 1.0119964 | 0.907666 | 1.061626 |
| N21546 | 1 | 0.832448 | 0.761707 | 0.898708 | 0.8271036 | 1.006878 | 0.925979 |
| N22776 | 1 | 1.040344 | 0.931029 | 1.141682 | 1.0343826 | 1.257707 | 1.028483 |
| N24059 | 1 | 0.865106 | 0.953974 | 0.854964 | 0.9528342 | 1.236834 | 1.148547 |
| N25240 | 1 | 0.941342 | 0.937738 | 1.082404 | 0.8980388 | 0.902085 | 0.940373 |
| N27190 | 1 | 0.877805 | 0.77018 | 0.943582 | 0.8502897 | 1.086685 | 0.859053 |
| N29545 | 1 | 1.007809 | 0.866036 | 1.050322 | 0.825753 | 0.928472 | 0.901554 |
| N30302 | 1 | 0.819778 | 0.815029 | 0.973156 | 0.7083085 | 1.084153 | 0.977039 |
| N32199 | 1 | 1.33909 | 1.162917 | 1.035487 | 1.1961197 | 0.923611 | 1.030052 |
| N32811 | 1 | 0.916485 | 0.984693 | 0.970755 | 0.9345574 | 0.992766 | 0.970876 |
| N33274 | 1 | 0.887141 | 1.002172 | 0.97505 | 1.0969407 | 1.148576 | 1.092061 |
| N36174 | 1 | 1.056902 | 1.098681 | 1.113014 | 0.9389056 | 1.093005 | 0.972182 |
| N39434 | 1 | 0.919748 | 0.850388 | 0.58723 | 0.9310085 | 1.086242 | 1.191679 |
| N46828 | 1 | 0.86447 | 0.878095 | 0.828239 | 0.7887738 | 1.050606 | 1.348479 |
| N48057 | 1 | 1.608615 | 1.665841 | 1.235445 | 1.7622408 | 0.882993 | 0.806706 |
| N49068 | 1 | 1.007441 | 1.04989 | 1.008703 | 0.9935802 | 0.958703 | 0.998915 |
| N49763 | 1 | 0.889168 | 0.853238 | 0.966149 | 0.767629 | 1.01695 | 0.998982 |
| N49774 | 1 | 1.241661 | 1.297971 | 1.193924 | 1.2699584 | 1.013818 | 0.811301 |
| N50963 | 1 | 1.004001 | 1.064504 | 1.194106 | 0.9727183 | 0.968513 | 0.869346 |
| N51002 | 1 | 0.798593 | 0.813465 | 0.790852 | 1.7568984 | 1.099077 | 1.076614 |
| N52765 | 1 | 0.918149 | 0.822779 | 0.88303 | 0.840794 | 0.910559 | 0.907224 |
| N52874 | 1 | 0.788358 | 0.802108 | 0.794038 | 0.7292356 | 1.204968 | 1.019876 |
| N56872 | 1 | 1.201696 | 1.401681 | 1.272075 | 1.2794394 | 0.939557 | 0.903124 |
| N57553 | 1 | 0.966091 | 0.989511 | 1.225205 | 1.0820507 | 1.99822 | 0.972768 |
| N59866 | 1 | 1.012147 | 1.142632 | 1.097583 | 1.1017736 | 0.929559 | 1.230973 |
| N62985 | 1 | 0.906856 | 0.942227 | 0.94655 | 0.8739335 | 1.091431 | 1.010901 |
| N63567 | 1 | 0.768424 | 0.816821 | 0.990367 | 0.7200704 | 1.117635 | 1.042884 |
| N63949 | 1 | 0.916085 | 0.87133 | 0.848224 | 0.8746091 | 0.992975 | 0.92618 |
| N63968 | 1 | 0.958879 | 0.973401 | 1.012219 | 0.9068127 | 0.862947 | 0.92255 |
| N64519 | 1 | 0.896825 | 0.838656 | 0.936961 | 0.7578984 | 0.984605 | 0.928146 |
| N64753 | 1 | 0.941264 | 0.97871 | 1.039666 | 0.7868757 | 1.153132 | 0.925666 |
| N66208 | 1 | 0.835756 | 0.941472 | 1.021665 | 0.9314328 | 1.006292 | 0.935932 |
| N66607 | 1 | 0.87292 | 0.68775 | 0.732023 | 0.73539 | 0.890822 | 0.897616 |
| N67634 | 1 | 0.878181 | 0.843084 | 0.834282 | 0.8611374 | 0.900717 | 0.856054 |
| N70057 | 1 | 1.069522 | 1.150563 | 0.887348 | 1.1796869 | 0.999088 | 0.961398 |
| N70088 | 1 | 0.846924 | 0.797955 | 0.857073 | 0.797757 | 1.060439 | 0.903721 |
| N70734 | 1 | 1.164483 | 1.06589 | 1.101264 | 1.2195266 | 1.057516 | 1.165911 |
| N70739 | 1 | 0.961436 | 0.957984 | 0.92841 | 0.9805164 | 1.06416 | 0.914697 |
| N71628 | 1 | 0.896976 | 0.854359 | 1.03818 | 0.7701471 | 1.022108 | 0.990365 |
| N71692 | 1 | 0.937273 | 1.003394 | 0.988476 | 0.9033707 | 0.990826 | 1.131455 |
| N73611 | 1 | 1.297565 | 1.41705 | 1.178316 | 1.5650768 | 0.881542 | 0.975904 |
| N73625 | 1 | 0.825903 | 0.846555 | 1.033397 | 0.7722116 | 0.874607 | 0.890221 |
| N73680 | 1 | 0.906119 | 0.925972 | 0.90404 | 0.9123425 | 0.948969 | 0.849401 |
| N78909 | 1 | 0.966201 | 1.044499 | 1.016265 | 1.0234429 | 1.049879 | 1.182214 |
| N91921 | 1 | 0.806504 | 0.846147 | 0.88057 | 0.7645259 | 1.0568 | 0.944893 |
| N92359 | 1 | 0.959611 | 0.98257 | 0.979786 | 0.9299476 | 0.951615 | 0.865062 |
| N92705 | 1 | 1.279024 | 1.168388 | 1.052677 | 1.22548 | 0.780887 | 1.083865 |
| N93214 | 1 | 1.121119 | 1.130417 | 1.439918 | 1.2424794 | 1.109485 | 1.103622 |
| N93582 | 1 | 1.011101 | 0.89336 | 0.614402 | 0.9394763 | 1.452517 | 1.292358 |
| N93686 | 1 | 1.325922 | 1.348708 | 1.333052 | 1.3415382 | 0.960468 | 1.007296 |
| N93695 | 1 | 0.822223 | 0.777303 | 0.820018 | 0.7925849 | 1.019021 | 0.931464 |
| N98485 | 1 | 1.232866 | 1.241368 | 1.238562 | 1.0632486 | 0.948871 | 0.943573 |
| R23148 | 1 | 0.954832 | 0.845738 | 0.878327 | 0.7929005 | 0.856186 | 1.017217 |
| R27776 | 1 | 1.033112 | 1.058374 | 0.970562 | 1.0366757 | 0.966882 | 1.100235 |
| R36571 | 1 | 0.710684 | 0.729999 | 0.734378 | 0.6432868 | 1.198846 | 1.241952 |
| R36571 | 1 | 0.694939 | 0.696449 | 1.606599 | 0.7393924 | 1.340359 | 1.181264 |
| R40212 | 1 | 0.942193 | 0.951873 | 0.774142 | 0.9494024 | 0.932835 | 0.899745 |
| R40212 | 1 | 1.016165 | 1.022446 | 1.09523 | 1.1107751 | 1.168317 | 1.064666 |
| R43509 | 1 | 0.847709 | 0.899676 | 0.733232 | 0.8911717 | 1.203215 | 1.172061 |
| R44769 | 1 | 0.871441 | 0.800363 | 0.905933 | 0.719597 | 0.926038 | 0.959207 |
| R51346 | 1 | 0.944174 | 0.976282 | 0.832669 | 1.0246392 | 0.95633 | 1.239615 |
| R51346 | 1 | 0.956551 | 1.023799 | 0.882332 | 1.1492948 | 1.110138 | 1.216308 |
| R52548 | 1 | 1.06543 | 1.100257 | 0.861554 | 1.1063478 | 0.835609 | 0.920735 |
| R52548 | 1 | 1.088231 | 1.04593 | 1.073169 | 1.1291551 | 0.936634 | 1.005849 |
| R56871 | 1 | 1.042064 | 1.138658 | 1.027573 | 1.025844 | 0.874662 | 0.82873 |
| R70888 | 1 | 1.131789 | 1.172097 | 0.991484 | 1.1189956 | 0.838385 | 1.092729 |
| R77239 | 1 | 0.883044 | 0.850112 | 1.096262 | 0.7407149 | 1.019721 | 0.945174 |
| R78521 | 1 | 1.064924 | 0.889554 | 0.601629 | 0.8631923 | 1.198597 | 1.049049 |
| R80779 | 1 | 0.930649 | 0.884424 | 0.94213 | 1.0296758 | 0.93633 | 0.866647 |
| T41077 | 1 | 1.013245 | 1.011068 | 1.089246 | 1.1012545 | 1.405832 | 1.166414 |
| T51539 | 1 | 1.107889 | 1.230775 | 1.158049 | 1.3079851 | 1.059862 | 1.067437 |
| T60120 | 1 | 1.548882 | 1.414645 | 1.593976 | 1.5650458 | 1.085648 | 1.183081 |
| T61071 | 1 | 1.142784 | 0.902267 | 1.074655 | 1.0815368 | 1.067098 | 1.102042 |
| T68859 | 1 | 0.825878 | 0.882696 | 0.892947 | 0.8489041 | 0.967572 | 0.947118 |
| T91080 | 1 | 0.842922 | 0.841714 | 0.858153 | 0.8479745 | 1.118368 | 1.270065 |
| W04645 | 1 | 0.835687 | 0.91544 | 0.673532 | 0.9336297 | 1.083401 | 1.14662 |
| W15274 | 1 | 1.134359 | 1.139448 | 1.053531 | 1.1802496 | 0.971526 | 1.191965 |
| W15305 | 1 | 1.555359 | 1.632855 | 1.232561 | 1.6857938 | 1.060665 | 1.031018 |
| W15542 | 1 | 0.997826 | 0.923665 | 0.943805 | 0.9218903 | 1.220525 | 1.056741 |
| W19228 | 1 | 1.015133 | 0.972852 | 1.059088 | 0.9574817 | 1.000248 | 0.936786 |
| W20438 | 1 | 0.976291 | 0.940526 | 0.962191 | 0.9131646 | 1.069214 | 1.212941 |
| W37338 | 1 | 0.862311 | 0.920465 | 0.818143 | 0.960439 | 1.356553 | 1.41395 |
| W37680 | 1 | 0.947389 | 0.969441 | 0.829567 | 0.9294019 | 0.931675 | 1.189392 |
| W51951 | 1 | 1.093566 | 1.141348 | 1.083594 | 1.1156364 | 0.969224 | 1.187485 |
| W53000 | 1 | 0.992134 | 0.958367 | 0.961489 | 1.0015315 | 0.78603 | 0.827304 |
| W60286 | 1 | 0.992076 | 1.094144 | 0.922999 | 0.9292585 | 0.902597 | 1.111503 |
| W61361 | 1 | 1.043006 | 1.114992 | 1.117691 | 1.2170024 | 1.061539 | 0.926067 |
| W63789 | 1 | 0.981516 | 0.845941 | 1.077012 | 0.9139095 | 1.198991 | 1.067644 |
| W70051 | 1 | 1.045484 | 1.077204 | 0.885613 | 1.196918 | 0.781189 | 0.832748 |
| W72227 | 1 | 1.051143 | 1.016763 | 0.993233 | 0.8801445 | 0.81081 | 0.916288 |
| W72525 | 1 | 0.913135 | 0.971114 | 1.103532 | 0.7959048 | 1.045279 | 0.910492 |
| W73634 | 1 | 0.853761 | 0.834342 | 0.818274 | 0.9536823 | 0.860474 | 0.849348 |
| W74123 | 1 | 0.870028 | 0.784221 | 0.885638 | 0.7949466 | 1.134668 | 1.084966 |
| W74725 | 1 | 0.7341 | 0.686407 | 0.635796 | 0.7043394 | 0.86558 | 0.78501 |
| W80692 | 1 | 1.027835 | 1.00171 | 0.967275 | 0.9912181 | 0.749192 | 0.92555 |
| W81432 | 1 | 0.855937 | 0.793676 | 0.708297 | 0.8486153 | 0.965392 | 0.896708 |
| W84868 | 1 | 0.969229 | 0.982291 | 1.093056 | 1.0757407 | 1.035915 | 0.973028 |
| W86423 | 1 | 1.042727 | 1.042383 | 1.091678 | 1.0626197 | 0.961833 | 0.807227 |
| W92233 | 1 | 1.072255 | 1.133704 | 0.896242 | 1.1153299 | 0.884367 | 1.228653 |
| W94136 | 1 | 1.022098 | 0.996403 | 0.959408 | 0.9458512 | 0.905271 | 1.008272 |
| W95082 | 1 | 1.391471 | 1.145307 | 1.415114 | 1.5629791 | 0.844365 | 0.824088 |
| W95428 | 1 | 1.04926 | 1.002801 | 0.95316 | 0.9982313 | 0.960531 | 0.954015 |
| W96205 | 1 | 0.901446 | 0.931021 | 0.993703 | 0.9089391 | 0.960368 | 0.973151 |
| W96450 | 1 | 0.814064 | 0.857257 | 0.671155 | 0.8386957 | 1.170378 | 1.086605 |
| W96463 | 1 | 0.913572 | 0.905833 | 0.810884 | 0.8457982 | 0.84104 | 0.801432 |

Many more established model systems (e.g., tumor cell to differentiated cell transitions, transdifferentiation between differentiated cell types, stem cell differentation to different specialized cells lineages) exist that can be studied using this approach and analyzed according to the invention in order to obtain standard reference trajectories. Another specific example of another model system is the induction of prostate cancer cells (LnCap) to transition to neuron-like differentiated cells by addition of cAMP and reversion of this process by removal of cAMP. The same approach to experimental design can be used with any system for which two stable cell behavioral states can be identified as well as stimuli that induce the cells to transition between these states.

### Example 2

### Gene Expression Profiling

According to the invention, cells that undergo a cell state transition can be used to characterize genome-wide gene expression profiles (or any other genome-wide molecular activities of the cell) during the entire switching time period for the cell state transitions. For example, for each transition described in the experimental HL60 system of Example 1, time-dependent change in genome-wide gene expression profiles may be monitored by analyzing mRNA expression levels on DNA microarrays (spotted cDNA on glass Research Genetics nylon filters, Affymetrix GeneChips) for 10-20 regularly spaced time points between the initial state and final state. In a typical set of experiments, there are approximately 10,000 gene expression values for 10 different time points (i.e. 10 profiles with 10,000 values each) for each transition process. These are stored electronically, thereby creating the first elements of a gene database. The same method may be carried out with different types of gene and protein arrays that permit simultaneous analysis of larger numbers of genes and, eventually, the entire genome.

### Example 3

### Application of Dynamic Network Analysis Algorithms

Conventional approaches typically compare only the initial and the final state of a cellular transition and identify genes that are differentially expressed. The genes whose expression correlates with the final state may be involved in mediating the transition. However, these genes more likely are “innocent bystanders” or are expressed as a consequence, rather than a cause, of the cellular transition. Other approaches monitor temporal profiles and cluster the genes based on similarity of their temporal response (i.e. that exhibit similar changes in expression at similar times). In contrast, methods of the invention analyze how the expression of thousands of genes change during the time-course of the entire cellular transition using a novel integrative approach that treats the time-evolution of the entire gene expression profile as an entity that reveals the dynamics of the underlying genetic network. Therefore, methods of the invention are able to detect genes that are operative in the transition process, including those that are turned off again once the transition has initiated (e.g., “toggle switch genes”) or genes that exhibit different temporal responses. Therefore, methods of the invention are particularly useful to identify “switch genes” of this type as well as any other genes or molecular activities that are causally involved in a cellular transition, but overlooked by conventional methods based on statistical correlation analysis.

Accordingly, methods of the invention are useful to identify dynamic gene activity signatures that are predictive for specific state transitions (e.g., A-F, as described above in Example 1), and specific genes that could be causally involved in triggering one of these cell state transitions (e.g., a switch from a stem cell precursor to a macrophage or granulocyte). Changes in expression of distinct sets of early molecular markers typically precedes the appearance of conventional individual biochemical markers of cell specialization and thus, these genes or a temporal series of genes can be used as early gene-based markers for stem cell lineage switching.

The predictive value of the information in the dynamic gene signatures identified according to the invention can be evaluated by comparing the trajectories between the different forward and reverse, and divergent and convergent state transitions (A-F). This comparison is useful to identify a minimal set of information (“as early as possible, with as few variables as possible”) necessary to reliably predict the various possible outcomes of a stem cell differentiation process.

An analysis of the data obtained from the genome-wide gene profiling according to Example 2 will also indicate a set of genes (approximately 100) that are activated within 2 days and are likely to be involved in the cellular switch (which may take up to 7 days to complete for the above examples A-F). Master genes that control the switching of a group of other genes that are responsible for this type of switching also can be identified. Candidate master genes, or set of genes (or their products) identified by methods of the invention can be ectopically activated using conventional molecular biology methods (e.g., via transfection and overexpression) to demonstrate their causative role in the cell transition process and thus, their potential value as drug targets. The functional importance of other genes identified using methods of the invention can be deduced from the analysis of the scientific literature. For example, c-fes is a gene which is already known to actively drive the switching between precursor cells and macrophages. Therefore, methods of the invention will identify the c-fes gene in addition to other genes as important master genes in the example of switching between granulocytes and their precursor cells.

### Example 4

### Collection of Dynamic Gene Signatures of Elementary Switches

The information relating to the temporal changes in all the gene expression levels during the various state transitions studied in Examples 1-3 are stored electronically. This information represents the seed for establishment of a “gene trajectory” database containing dynamic gene signatures for elementary biological cell state transition processes—in the case of Example 1, stem cell differentiation. Because of the way in which cells process information in a molecular signaling network, their behavior is highly constrained and obeys distinct rules imposed by the network dynamics. Thus, for any genome, there is a finite set of canonical elementary processes for each cell type which determines how and when a cell becomes functionally active—divides, dies, migrates, differentiates, etc.—as well as the different potential behavioral states the cell may assume. Cell type-specific databases have huge commercial value because within them are contained all of the sequential, time-dependent changes in specific gene activities and associated gene and protein targets that mediate functional control of each particular cell type studied and characterized with these methods. Since the dynamic signatures represent a novel type of functional genomics data, new database structures may be developed. The data obtained in Examples 1-3 serve as a “toy data set” for designing and testing the database structure as well as optimizing the information retrieval algorithms. In this example, a small database is developed containing the dynamic trajectory signatures (in gene expression state space of 6000 genes) involved in stem cell switching in HL60 cells. A useful prototype relational database has a predetermined structure for storing and retrieving the high-dimensional dynamic signature data. The individual genes can be annotated with other known functional properties available in public gene databases or be hyperlinked to the latter. In this manner, a preferred database is created that is designed to accommodate future data relating to an expanding set of dynamic signatures for other biological behaviors, disease processes, and cell types.

### Example 5

### Selection of Drug Candidates for Animal Testing

The cell fate transition processes of the multipotent HL60 precursor cells studied in Examples 1-4 represent a medically-relevant model of cell behavior. Thus, the studies described above are useful examples of how to directly deliver information relating to a set of genes that represent potential molecular targets for therapeutic intervention in leukemia as well as design criteria for developing novel drugs to promote growth and expansion of specific lymphoid cell lineages.

The results obtained in Examples 1-4 can be utilized for preclinical drug discovery by exploiting the information in the trajectory databases in various ways including functional screening of drug candidates.

Drug Target Identification.

The invention also provides specific information on individual genes and sets of genes that are likely to be causative in the switch between different cell behavior states, such as in the transition of lymphoid cancer cells (e.g., leukemia cells) into a quiescent, differentiated, and hence, non-malignant phenotype. The direct contribution of these genes to the switch between attractors will be indicated by their direct contribution to the change in the form (e.g., ‘elbow’) of the phase portrait. Genes that are causally involved in this process are candidate drug targets for development of novel differentiation therapeutics. Starting from this set of candidate target genes, therapeutic molecules can be developed through de novo molecular drug design, or from compound libraries and tested in vitro. One aspect of the invention, as exemplified above, is to analyze the reversibility of cell transition processes, and the conditions under which such reversion of cell state switching occurs. Surprisingly, some of the above paths are bidirectional in that the transition process reverses when the stimulus is removed. Other than providing additional trajectory information, the bidirectionality of these transitions can be exploited for therapeutic interference. Since it is generally much easier to develop compounds that inhibit rather than activate genes or proteins, genes that control the reverse transition (from a differentiated to a cancerous state) and are also present in a dynamic gene signature database can be exploited to identify specific genes whose inhibition will destabilize the proliferative state and thereby promote differentiation and reversal of the malignant state.

In one aspect of the invention, a drug target is identified as one or more molecules that are important contributors to a cellular process associated with a disease. Such molecules or networks of molecules are identified by first generating a reference dynamic signature or phase portrait representative of the cellular process, using data for a large set of molecules (for example cell-wide gene or protein expression data, gene or protein modification data, lipid data, or other biological data relating to a large set of cellular molecules or molecular events). Subsequently, a dynamic signature or phase portrait is generated using data for a subset of the molecules that were used to generate the reference dynamic signature or phase portrait. This second dynamic signature or phase portrait is compared to the reference using, for example, a morphometric index, an area under the curve analysis, a pattern recognition algorithm, or other comparison method. If the comparison reveals significant differences, the subset of molecules does not contain all of the molecules that are important contributors to the cellular process. In contrast, if the second dynamic signature or phase portrait is similar to the reference, the subset of molecules contains the important contributors to the cellular process. Similarly, additional subsets of molecules can be evaluated to determine whether they contain important molecules for the cellular process. This analysis can be repeated in an iterative fashion until the core molecular components of a cellular process are identified. These components can then be used as targets for drug development and therapeutic response or for toxicological testing.

According to the invention, subsets of data described above can be chosen randomly, or chosen based on dimensionality reduction of the original data set using clustering methods or principal component analysis, or chosen as a subset of a previously used subset.

In one embodiment, based on their relative contribution to a dynamic signature or phase portrait, different molecules or molecular events can be categorized or ranked as a function of their relative importance in a given cellular process. This provides a hierarchy of targets for drug design or for prioritizing lead drug candidates for further development and testing.

Functional Screening of Drug Candidates

In one embodiment of the invention, molecules identified as described above can be used to screen for drug candidates and to identify lead compounds in drug development programs. In one aspect, such molecules can be used as drug targets in drug development. Alternatively, dynamic signatures or phase portraits based on one or few critical molecules involved in a disease process, such as cancer development, cancer progression or cancer regression, can be used to monitor the effectiveness of drug candidates in treating a disease and obtaining a desired cellular outcome. The desired cellular outcome can be represented by a reference representation (e.g. a dynamic signature or phase portrait) to which the effect of a candidate drug treatment can be compared. In another embodiment of the invention, identified dynamic gene signatures that represent temporal changes in expression of particular genes early in a transition process can serve as surrogate markers for identification of drugs that induce this switching process. For example, these gene trajectories provide a means to screen chemical compound libraries for agents that specifically induce a particular state transition (e.g., development of granulocytes from stem cells). The trajectory information allows candidate agents to be evaluated even before the cellular transition is complete, that is, before conventional molecular markers indicative of the completed process (e.g., new cell surface antigens) are expressed. Therefore, the invention provides methods that can greatly accelerate the drug discovery process, especially when the process under study normally takes many days to complete (e.g., differentiation). In this case, the specific molecular target of the compound need not be known; it is the expression of a particular dynamic signature of gene expression profiles that is used as the marker for functional activity.

This dynamic signature-based approach permits systematic development of “multi-target” drugs based on a biological rationale. This is important because most highly effective drugs in use today and that were discovered based on serendipity act on multiple targets. Yet, the current paradigm of rational drug development is still limited to targeting single molecular targets. According to the invention, dynamic gene signatures can be exploited to identify this type of multi-target drug. For example, the dynamic signatures obtained from analysis of the HL-60 precursor cell system can be used to screen for novel drugs that treat human leukemia by modifying the cellular phenotype and inducing differentiation and quiescence, rather than killing the growing cells.

### Example 6

### Phase Portraits of Fibroblast Proliferation and Hematopoietic Differentiation

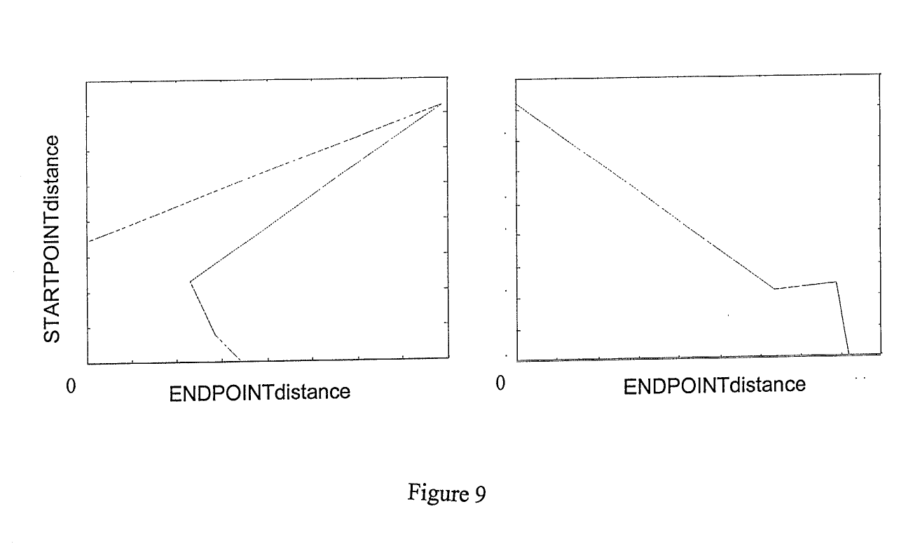

The relative extent that an “elbow” peak deviates towards higher values and away from the monotonic diagonal path of a phase portrait appears to correlate with the constraints which are imposed by the network architecture on that transition and which had to be overcome to produce the cell state transition. For example, the switch from growth to differentiation generates smaller deviations in the phase portrait than the switch from quiescence to growth (FIG. 9). This finding reflects the observation that it is ‘easier’ to push the cell into the quiescent, differentiated state (a process that often occurs spontaneously) than to trigger the entry into the growth state from a quiescent state (which requires specific growth factors). FIG. 9 shows the analysis of published gene expression data from living fibroblast cells stimulated to proliferate (left) versus hematopoietic cells stimulated to differentiate (right). Note the higher peak deviation in the case of growth stimulation.

The data used for FIG. 9 have been published in the context of a conventional analysis that clustered genes according to the similarity of the time course of individual genes in response to the perturbation (Iyer et al, 1999; Tamayo et al., 2000). However, these data were not analyzed using a dynamic representation or algorithm of the present invention. Nonetheless, although the underlying experiments were not optimally designed for a dynamic network analysis of the cell state according to the invention, they still demonstrate the basic utility of the present invention.

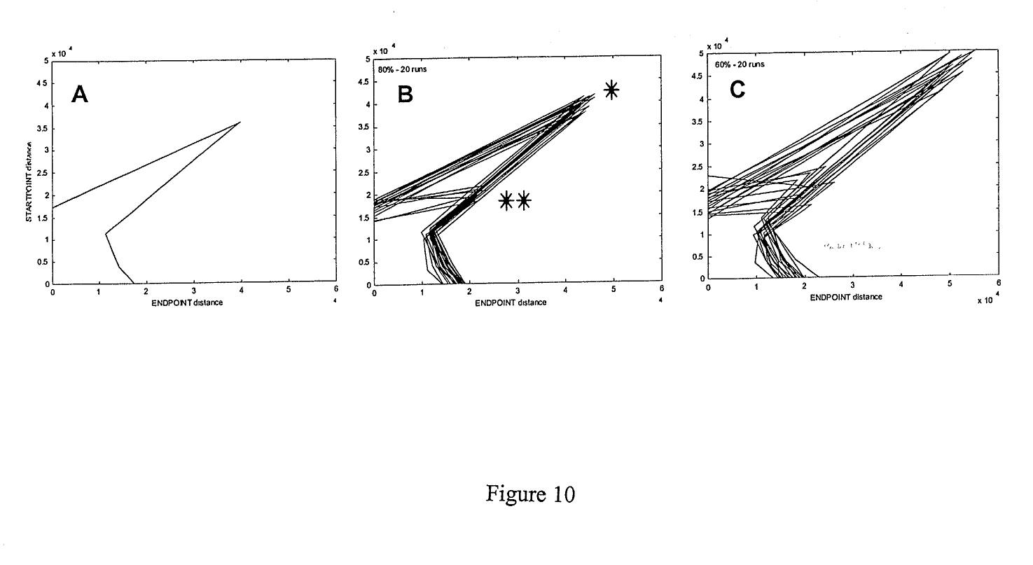

The robustness of the phase portrait representation of FIG. 9 can be evaluated by performing the same analysis on subsets of the published gene information used to generate FIG. 9. To show the robustness and the contribution of individual genes to the phase portrait, randomly chosen subsets of 80% or 60% of the approximately 6000 genes used in the original analysis were used to generate additional phase portraits. The analysis was run 20 times with different randomly selected subsets of the genes and the resulting phase portraits were overlaid as shown in FIG. 10. Panel A shows the original analysis. Panels B and C show analyses using subsets of 80% and 60% of the genes, respectively. Increased “blurring” of the phase portrait is observed as the percentage of genes used in the analysis is decreased from 80% to 60%. In addition, reducing the gene number also revealed two classes of phase portraits: one with high “elbow” (* in panel B) and one with low “elbow” (** in panel B). This finding indicates that the randomly selected subsets either leave out or include one or more critical genes that are mechanistically relevant in that they are in part responsible for the high elbow.

### Example 7

### Algorithms and Databases

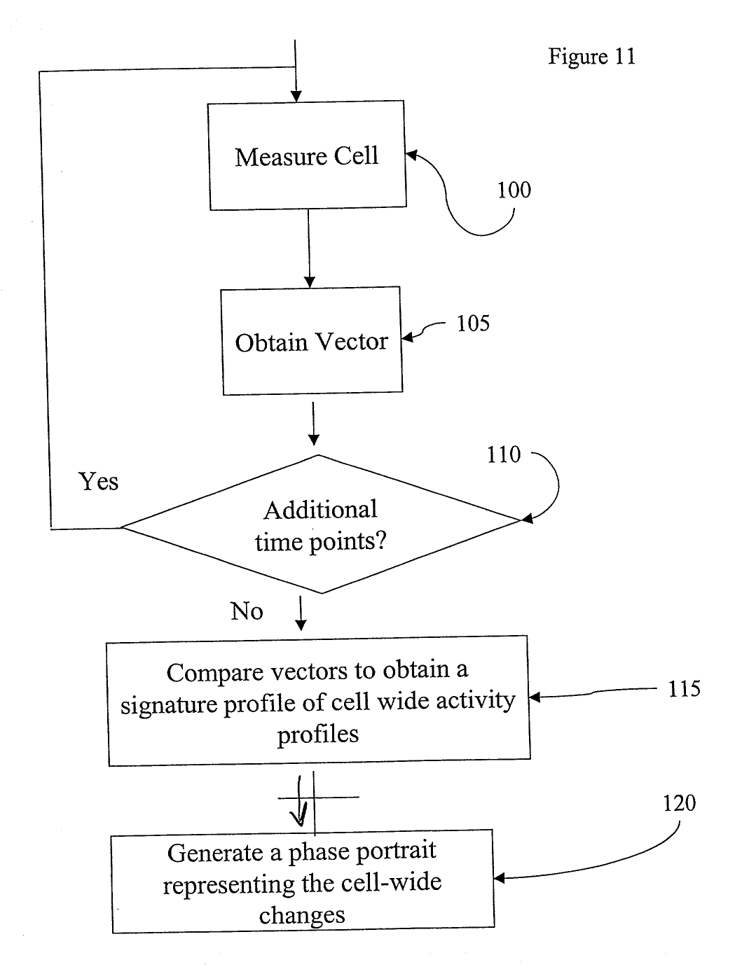

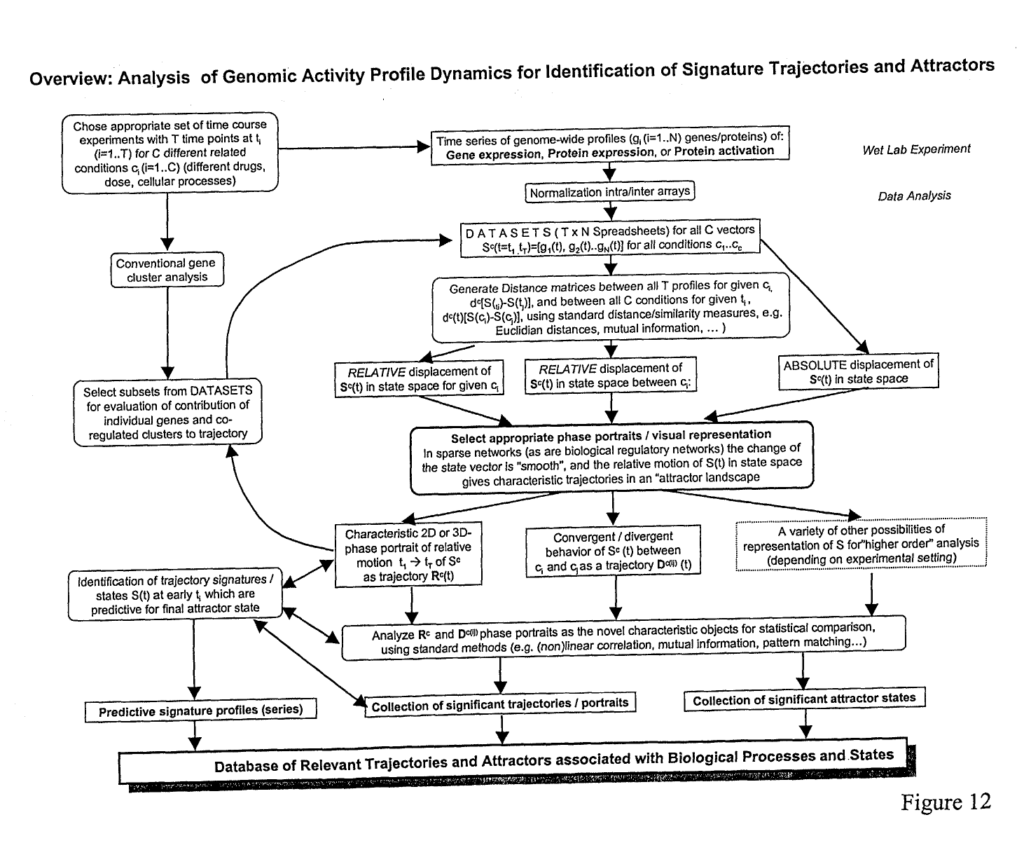

The invention provides algorithms for simplifying the analysis of the large number of molecular components of a cellular event. An outline of an analysis algorithm of the invention is shown in FIG. 11. FIG. 12 shows a more detailed version and provides examples of database related aspects of the invention. As discussed above, the invention provides methods for generating useful representations of cellular processes, for example dynamic signatures and phase portraits. According to the invention, such representations, including dynamic signatures, phase portraits, predictive signature profiles, collections of significant trajectories/portraits, and collections of significant attractor states can be stored electronically on a database, and accessed for subsequent use, as indicated in FIG. 12. In one embodiment, the database stores data structures with other information, including information related to cellular processes, cell switches, attractors, drug treatments, drug structures, drug effectiveness, diagnoses, and disease progression. In a preferred embodiment, a database contains patient specific information, for example information relating to a patient's family or medical history, responses to drug treatments, disease progression, and genetic information such as RFLP or polymorphism data. Accordingly, the information can be accessed and searched in order to correlate a representation of the invention with useful information. For example, a dynamic signature or phase portrait obtained for one or more patients for a given cellular process can be compared to dynamic signatures or phase portraits stored on the database to determine whether there is a reference signature or portrait on the database that is sufficiently similar to provide a diagnosis for a given disease or condition, or to provide one or more patients with a disease prognosis. Additional information relating to suggested or recommended therapies, including drug treatments, is preferably located and accessed on a database of the invention, in association with or linked to the corresponding dynamic signature or phase portrait. In addition, information relating to cellular processes can be accessed and used to identify targets for drug screening or in assays for drug evaluation as discussed above. For example, a dynamic signature can be generated for one or more drugs in a screening assay. Each dynamic signature can be compared to one or more reference dynamic signatures to determine whether the drug being tested results in a dynamic signature characteristic of a desired drug treatment outcome.

According to preferred embodiments of the invention, information can be stored on a computer system with a memory, a processor, an input/output interface, and a removable data medium, all linked by a bus. The memory can be a RAM, ROM, CDROM, Tape, Disk, or other form of memory. The removable data medium can be a magnetic disk, a CDROM, a tape, an optical disk, or other form of removable data medium. Accordingly, dynamic signatures, phase portraits, and related information is preferably stored in a memory in a computer system, or alternatively in a removable data medium such as a magnetic disk, a CDROM, a tape, or an optical disk. In a preferred embodiment, information is stored on a computer system including two or more networked computers. In a further embodiment, the input/output of the computer system can be attached to a network and information about cellular activity profiles, signatures and portraits can be accessed and/or transmitted across the network. For example, information can be accessed and/transmitted on a web-based system using a web browser running on a workstation. According to preferred embodiments of the invention, methods for analyzing cell profile information and for generating representations of cellular processes such as dynamic signatures and phase portraits are implemented on a computer system, such as a computer system described above.

Although the present invention has been described with reference to specific details, it is not intended that such details should be regarded as limitations upon the scope of the invention, except as and to the extent that they are included in the accompanying claims.

The disclosure of each of the patent documents and scientific publications disclosed herein, is incorporated by reference into this application in its entirety.

1. A method for representing a change in cellular activity, the method comprising the steps of:
(a) measuring a cellular activity profile at each of a plurality of time points during a cellular process;
(b) assigning a cell-state vector to each of the cellular activity profiles; and,
(c) generating from said cell-state vectors a dynamic signature representing a trajectory in state-space of the cellular process.

2. A method for predicting the behavior of a cellular material, the method comprising the steps of:
(a) measuring a cellular activity profile at each of a plurality of time points;
(b) assigning a cell-state vector to each of the cellular activity profiles;
(c) generating from said cell-state vectors a dynamic signature representing a trajectory in state-space of the cellular process; and,
(d) comparing said dynamic signature to a reference dynamic signature to predict cell behavior based on a reference cellular process represented by said reference dynamic signature.

3. The method of claim 2, further comprising the step of providing a disease diagnosis to a patient.

4. The method of claim 2, further comprising the step of providing a disease prognosis to a patient.

5. The method of claim 2, further comprising the step of recommending a therapy to a patient.

6. The method of claim 1 or 2, wherein step (c) comprises the steps of:
i. calculating a distance between each of said cell-state vectors and a reference vector; and, obtaining a phase portrait of said cellular process by plotting each of the cell-state vectors as a function of said calculated distances, wherein the axes for the phase portrait are chosen in each case to be most informative.

7. The method of claim 6 further comprising the step of
iii. obtaining a temporal profile of the distance between the state vectors of two or more processes.

8. The method of claim 6, wherein said reference vector is the same for each of said cell-state vectors.

9. The method of claim 6, wherein said reference vector is a cell-state vector.

10. The method of claim 6, comprising the step of generating a matrix of distances between each of said cell-state vectors.

11. The method of claim 1 or 2, wherein said cellular activity profile is a gene expression profile.

12. The method of claim 1 or 2, wherein said cellular activity profile is a protein expression profile.

13. The method of claim 1 or 2, wherein said cellular activity profile is a protein activation profile.

14. The method of claim 9, wherein said protein activation profile is selected from the group consisting of a profile of protein activation by covalent or non-covalent post-translational modification, and a profile of protein subcellular localization.

15. The method of claim 1 or 2, wherein said cellular activity is measured by assaying levels of cellular molecules selected from the group consisting of lipids, nucleotides, carbohydrates, and metabolic intermediates.

16. The method of claim 1 or 2, wherein said cellular activity profile is an activity profile of between 10 and 100,000 genes or gene products.

17. The method of claim 15, wherein said cellular activity profile is an activity profile of between 100 and 30,000 genes or gene products.

18. The method of claim 1 or 2, wherein said cellular process is a transition from an initial cell state to a final cell state.

19. The method of claim 18, wherein said cellular activity profile is measured for said initial cell state and said final cell state.

20. The method of claim 1 or 2, wherein said plurality of time points comprises more than two time points during said cellular process.

21. The method of claim 1 or 2, wherein said cellular activity profile is continuously monitored.

22. The method of claim 1 or 2, wherein the said cellular process is triggered by a perturbation selected from the group consisting of a chemical, a biomolecule, genetic manipulation, irradiation, mechanical force, a toxin, and temperature change.

23. The method of claim 17 wherein the said perturbation is exerted at a strength between a subthreshold strength and a saturating strength.

24. The method of claim 13, wherein either one or both of said initial cell state and said final cell state is an attractor state.

25. The method of claim 1 or 2, wherein said time points represent intermediate states of said cellular process.

26. The method of claim 1 or 2, wherein said plurality of time points represent intermediate states of a disease process.

27. The method of claim 13, wherein said initial and said final cell states are independently selected from the group consisting of functional, quiescent, proliferating, differentiated, motile, contractile, secretory, activated, apoptotic, diseased, drug induced, toxin induced, genetically induced, and environmentally induced cell states.

28. The method of claim 1 or 2, wherein each of said cell-state vectors represents the position of the cell in functional gene activity state space.

29. The method of claim 5, wherein said distances are selected from the group consisting of Hamming distances, Minkowski metrics, linear correlation measures, non-linear correlation measures, Pearson correlations, dot products, Euclidian distances, squared Euclidian distances, rank correlations, and mutual information.

30. The method of claim 5, wherein said distances are plotted in a 2-dimensional graph.

31. The method of claim 30, wherein said 2-dimensional graph includes an axis that represents a variable selected from the group consisting of
(i) a distance to an initial cell state;
(ii) a distance to a final cell state;
(iii) a distance to previous states of the process separated by a defined time period;
(iv) a distances to reference cell states;
(v) a distance to cell states in the same or other cellular processes; and,
(vi) a time evolution of the cellular process.

32. The method of claim 2, wherein said distances are plotted in a 3-dimensional graph.

33. The method of claim 1 or 2, wherein said cellular activity profile is measured in a cell culture, tissue culture, tissue or organ, or organism.

34 A method for identifying important molecular components of a cellular process, the method comprising the steps of:
(a) measuring a cellular activity profile at each of a plurality of time points during a cellular process; wherein each of said cellular activity profiles comprises a value for each of a plurality of molecular components;
(b) assigning a first cell-state vector to each of said cellular activity profiles, wherein each of said first cell-state vectors is derived from the values for the molecular components at a corresponding time point;
(c) assigning a second cell-state vector to each of said cellular activity profiles, wherein each of said second cell-state vectors is derived from the values for a subset of the molecular components at a corresponding time point;
(d) comparing a second dynamic signature generated from said second cell-state vectors with a first dynamic signature generated from said first cell-state vectors, thereby to determine whether the subset of molecular components contributes to the first dynamic signature representatvie of the cellular process.

35. The method of claim 34, further comprising the steps of
(e) generating one or more additional dynamic signatures based on values for one or more additional subsets of molecular components;
(f) comparing each of said additional dynamic signatures to said first dynamic signature, thereby to identify molecular components that contribute to a dynamic signature that is representative of the cellular process.

36. The method of claim 35, wherein said cellular process is a disease process.

37. The method of claim 36, wherein said disease process is selected from the group consisting of transformation, differentiation, and cancer progression.

38. The method of claim 35, wherein the molecular components identified in step (f) are screened as drug target candidates.

39. The method of claim 34, wherein the molecular components are selected from the group consisting of genes, proteins, lipids, nucleotides, carbohydrates, and metabolic intermediates.

40. The method of claim 34, wherein said subset of molecular components is chosen using a method selected from the group consisting of random selection, dimensionality reduction, clustering methods, and principal component analysis.

41. The method of claim 34, wherein an identified molecular component is a drug target.

42. A method for assaying a candidate drug, the method comprising the step of comparing a reference dynamic signature generated in the absence of drug candidate with a test dynamic signature generated in the presence of a drug candidate, wherein each of said dynamic signatures is generated based on a predetermined set of molecular components, thereby to determine whether said drug candidate alters a cellular process.

43. A method for monitoring a cellular process comprising the step of comparing a first dynamic signature to a reference dynamic signature, wherein each of said dynamic signatures is generated based on a predermined set of molecular components; thereby to determine the status of a cellular process.

44. The method of claim 42 or 43, wherein said cellular process is selected from the group consisting of toxicity, disease progression, and therapeutic response.
